SEQUENCE LISTING (7 sequences)

SEQ ID NO: 1 - DNA, 5009 bp, Homo sapiens
gcggcggcagcggcggcggtgatggctcctccgggctgcccgggttcgtgccccaacttcgccgtagtctgctccttcttggagcgctacgggccgctgctagacctgcctgagttgccgttccctgagctggagcgggtgctgcaggcgccgccgccggacgtcggcaacggagaagtaccaaaagaattggtggagctccatttgaagctgatgaggaaaattggcaaatctgttactgcagacagatgggaaaaatatttgatcaagatatgccaagagtttaacagtacctgggcatgggagatggagaagaagggctatcttgaaatgagtgttgaatgcaaactagcactcttaaagtacctctgtgagtgtcagtttgatgacaatctcaaattcaagaatattattaatgaggaggatgccgatactatgcgtctccagccaattggtcgagacaaagatggcctcatgtactggtaccaattggatcaagatcacaatgtcagaatgtacatagaagaacaagatgatcaagatggctcttcatggaaatgcattgtcagaaatcgaaacgagttggctgagactcttgcactcctgaaagcacaaattgatcctgtactattgaaaaactctagccaacaagacaactcttctcgggaaagtcccagcttagaggatgaggagactaaaaaagaggaagaaacacctaaacaagaggaacagaaagaaagtgaaaagatgaaaagtgaggagcagcctatggatttagaaaaccgttctacagccaatgttctagaagagactactgtgaaaaaagaaaaagaagatgaaaaggaacttgtgaaactgccagtcatagtgaagctagaaaaacctttgccagaaaatgaagaaaaaaagattatcaaagaagaaagtgattccttcaaggaaaatgtcaaacccattaaagttgaggtgaaggaatgtagagcagatcctaaagataccaaaagtagcatggagaagccagtggcacaggagcctgaaaggatcgaatttggtggcaatattaaatcttctcacgaaattactgagaaatctactgaagaaactgagaaacttaaaaatgaccagcaggccaagataccactaaaaaaacgagaaattaaactgagtgatgattttgacagtccagtcaagggacctttgtgtaaatcagttactccaacaaaagagtttttgaaagatgaaataaaacaagaggaagagacttgtaaaaggatctctacaatcactgctttgggtcatgaagggaaacagctggtaaatggagaagttagtgatgaaagggtagctccaaattttaagacagaaccaatagagacaaagttttatgagacaaaggaagagagctatagcccctctaaggacagaaatatcatcacggagggaaatggaacagagtccttaaattctgtcataacaagtatgaaaacaggtgagcttgagaaagaaacagcccctttgaggaaagatgcagatagttcaatatcagtcttagagatccatagtcaaaaagcacaaatagaggaacccgatcctccagaaatggaaacttctcttgattcttctgagatggcaaaagatctctcttcaaaaactgctttatcttccaccgagtcgtgtaccatgaaaggtgaagagaagtctcccaaaactaagaaggataagcgcccaccaatcctagaatgtcttgaaaagttagagaagtccaaaaagacttttcttgataaggacgcacaaagattgagtccaataccagaagaagttccaaagagtactctagagtcagaaaagcctggctctcctgaggcagctgaaacttctccaccatctaatatcattgaccactgtgagaaactagcctcagaaaaagaagtggtagaatgccagagtacaagtactgttggtggccagtctgtgaaaaaagtagacctagaaaccctaaaagaggattctgagttcacaaaggtagaaatggataatctggacaatgcccagacctctggcatagaggagccttctgagacaaagggttctatgcaaaaaagcaaattcaaatataagttggttcctgaagaagaaaccactgcctcagaaaatacagagataacctctgaaaggcagaaagagggcatcaaattaacaatcaggatatcaagtcggaaaaagaagcccgattctccccccaaagttctagaaccagaaaacaagcaagagaagacagaaaaggaagaggagaaaacaaatgtgggtcgtactttaagaagatctccaagaatatctagacccactgcaaaagtggctgagatcagagatcagaaagctgataaaaaaagaggggaaggagaagatgaggtggaagaagagtcaacagctttgcaaaaaactgacaaaaaggaaattttgaaaaaatcagagaaagatacaaattctaaagtaagcaaggtaaaacccaaaggcaaagttcgatggactggttctcggacacgtggcagatggaaatattccagcaatgatgaaagtgaagggtctggcagtgaaaaatcatctgcagcttcagaagaggaggaagaaaaggaaagtgaagaagccatcctagcagatgatgatgaaccatgcaaaaaatgtggccttccaaaccatcctgagctaattcttctgtgtgactcttgcgatagtggataccatactgcctgccttcgccctcctctgatgatcatcccagatggagaatggttctgcccaccttgccaacataaactgctctgtgaaaaattagaggaacagttgcaggatttggatgttgccttaaagaagaaagagcgtgccgaacgaagaaaagaacgcttggtgtatgttggtatcagtattgaaaacatcattcctccacaagagccagacttttctgaagatcaagaagaaaagaaaaaagattcaaaaaaatccaaagcaaacttgcttgaaaggaggtcaacaagaacaaggaaatgtataagctacagatttgatgagtttgatgaagcaattgatgaagctattgaagatgacatcaaagaagccgatggaggaggagttggccgaggaaaagatatctccaccatcacaggtcatcgtgggaaagacatctctactattttggatgaagaaagaaaagaaaataaacgaccccagagggcagctgctgctcgaaggaagaaacgccggcgattaaatgatctggacagtgatagcaacctggatgaagaagagagcgaggatgaattcaagatcagtgatggatctcaagatgagtttgttgtgtctgatgaaaacccagatgaaagtgaagaagatccgccatccaatgaggacagtgacactgacttttgtagccgtagactgaggcgacacccctctcggccaatgaggcagagcaggcgtttgcgaagaaagaccccaaagaaaaaatattccgatgatgatgaagaggaggaatctgaggagaatagtagagactctgaaagtgacttcagtgatgattttagtgatgattttgtagaaactcggcgaaggcggtcaaggagaaatcagaaaagacaaattaactacaaagaagactcagaaagtgacggttcccagaagagtttgcgacgtggtaaagaaataaggcgagtacacaagcgaagactttccagctcagagagtgaagagagctatttgtccaagaactctgaagatgatgagctagctaaagaatcaaagcggtcagttcgaaagcggggccgaagcacagacgagtattcagaagcagatgaggaggaggaggaagaggaaggcaaaccatcccgcaaacggctacaccggattgagacggatgaggaggagagttgtgacaatgctcatggagatgcaaatcagcctgcccgtgacagccagcctagggtcctgccctcagaacaagagagcaccaagaagccctaccggatagaaagtgatgaggaagaggactttgaaaatgtaggcaaagtggggagcccattggactatagcttagtggacttaccttcaaccaatggacagagccctggcaaagccattgagaacttgattggcaagcctactgagaagtctcagacccccaaggacaacagcacagccagtgcaagcctagcctccaatgggacaagtggtgggcaggaggcaggagcaccagaagaggaggaagatgagcttttgagagtgactgaccttgttgattatgtctgtaacagtgaacagttataagactttttttccatttttgtgctaatttattccacggtagctctcacaccagcgggccagttattaaaagctgtttaatttttcctagaaaactccactacagaatgacttttagaagaaaaatttcaacaaatcctgaagtctttctgtgaagtgaccagttctgaactttgaagataaataattgctgtaaattccttttgattttctttttccaggttcatggtccttggtaatttcattcatggaaaaaaatcttattataataacaacaaagatttgtatatttttgactttatatttcctgagctctcctgactttgtgaaaaagggtggatgaaaatgcattccgaatctgtgagggcccaaaacagaatttaggggtgggtgaaagcacttgtgctttagctttttcatattaaatatatattatatttaaacattcatggcatagatgatgatttacagacaatttaaaagttcaagtctgtactgttacagtttgagaattgtagataacatcatacataagtcatttagtaacagcctttgtgaaatgaacttgtttactattggagataaccacacttaataaagaagagacagtgaaagtaccatcataattaacctaaatttttgttatagcagagtttcttgtttaaaaaaaaataaaatcatctgaaaagcaaaaa

SEQ ID NO: 2 - protein, 1441 aa, Homo sapiens
MATAAAAAAVMAPPGCPGSCPNFAVVCSFLERYGPLLDLPELPFPELERVLQAPPPDVGNGEVPKELVELHLKLMRKIGKSVTADRWEKYLIKICQEFNSTWAWEMEKKGYLEMSVECKLALLKYLCECQFDDNLKFKNIINEEDADTMRLQPIGRDKDGLMYWYQLDQDHNVRMYIEEQDDQDGSSWKCIVRNRNELAETLALLKAQIDPVLLKNSSQQDNSSRESPSLEDEETKKEEETPKQEEQKESEKMKSEEQPMDLENRSTANVLEETTVKKEKEDEKELVKLPVIVKLEKPLPENEEKKIIKEESDSFKENVKPIKVEVKECRADPKDTKSSMEKPVAQEPERIEFGGNIKSSHEITEKSTEETEKLKNDQQAKIPLKKREIKLSDDFDSPVKGPLCKSVTPTKEFLKDEIKQEEETCKRISTITALGHEGKQLVNGEVSDERVAPNFKTEPIETKFYETKEESYSPSKDRNIITEGNGTESLNSVITSMKTGELEKETAPLRKDADSSISVLEIHSQKAQIEEPDPPEMETSLDSSEMAKDLSSKTALSSTESCTMKGEEKSPKTKKDKRPPILECLEKLEKSKKTFLDKDAQRLSPIPEEVPKSTLESEKPGSPEAAETSPPSNIIDHCEKLASEKEVVECQSTSTVGGQSVKKVDLETLKEDSEFTKVEMDNLDNAQTSGIEEPSETKGSMQKSKFKYKLVPEEETTASENTEITSERQKEGIKLTIRISSRKKKPDSPPKVLEPENKQEKTEKEEEKTNVGRTLRRSPRISRPTAKVAEIRDQKADKKRGEGEDEVEEESTALQKTDKKEILKKSEKDTNSKVSKVKPKGKVRWTGSRTRGRWKYSSNDESEGSGSEKSSAASEEEEEKESEEAILADDDEPCKKCGLPNHPELILLCDSCDSGYHTACLRPPLMIIPDGEWFCPPCQHKLLCEKLEEQLQDLDVALKKKERAERRKERLVYVGISIENIIPPQEPDFSEDQEEKKKDSKKSKANLLERRSTRTRKCISYRFDEFDEAIDEAIEDDIKEADGGGVGRGKDISTITGHRGKDISTILDEERKENKRPQRAAAARRKKRRRLNDLDSDSNLDEEESEDEFKISDGSQDEFVVSDENPDESEEDPPSNDDSDTDFCSRRLRRHPSRPMRQSRRLRRKTPKKKYSDDDEEEESEENSRDSESDFSDDFSDDFVETRRRRSRRNQKRQINYKEDSESDGSQKSLRRGKEIRRVHKRRLSSSESEESYLSKNSEDDELAKESKRSVRKRGRSTDEYSEADEEEEEEEGKPSRKRLHRIETDEEESCDNAHGDANQPARDSQPRVLPSEQESTKKPYRIESDEEEDFENVGKVGSPLDYSLVDLPSTNGQSPGKAIENLIGKPTEKSQTPKDNSTASASLASNGTSGGQEAGAPEEEEDELLRVTDLVDYVCNSEQL

SEQ ID NO: 3 - RNA, 21 nt, Artificial Sequence
  Description of Artificial Sequence Synthetic oligonucleotide
ggaaagacaucucuacuauuu

SEQ ID NO: 4 - RNA, 21 nt, Artificial Sequence
  Description of Artificial Sequence Synthetic oligonucleotide
uaaaugaucuggacagugauu

SEQ ID NO: 5 - RNA, 21 nt, Artificial Sequence
  Description of Artificial Sequence Synthetic oligonucleotide
ggacuuaccuucaaccaauuu

SEQ ID NO: 6 - DNA, 76 bp, Artificial Sequence
  Description of Artificial Sequence Synthetic oligonucleotide
ggatcccgtctttgtctcgaccaattggcttgatatccggccaattggtcgagacaaagattttttccaaaagctt

SEQ ID NO: 7 - DNA, 75 bp, Artificial Sequence
  Description of Artificial Sequence Synthetic oligonucleotide
ggatcccgatatcaattccttctgtccattgatatccgtggacagaaggaattgatatcttttttccaaaagctt

(19) United States
(12) Patent Application Publication
(10) Pub. No.: US 2007/0280969 A1
(43) Pub. Date: Dec. 6, 2007

(54) RSF-1 as a prognostic marker and therapeutic target

(75) Inventors: Ie-Ming Shih, Baltimore, MD (US); Tian-Li Wang, Baltimore, MD (US)

Correspondence Address: EDWARDS ANGELL PALMER & DODGE LLP;Client: JHU, P.O. BOX 55874, BOSTON, MA, 02205, US

(73) Assignee: The Johns Hopkins University, Baltimore, MD (US)

(21) Appl. No.: 11/604,183

(22) Filed: Nov. 24, 2006

Related U.S. Application Data
(60) Provisional application No. 60/739,117, filed on Nov. 23, 2005.
(60) Provisional application No. 60/751,962, filed on Dec. 20, 2005.

(51) Int. Cl. G01N 33/574; A61K 39/00; A61P 35/00; C12Q 1/18; C12Q 1/68

(52) U.S. Cl. 424/277.1; 435/32; 435/6; 435/7.23

(57) ABSTRACT

The instant invention provides cancer markers and methods of prognosis and diagnosis based on detection and/or quantitation of these markers. The invention further provides compositions for the treatment of cancer.

### RELATED APPLICATIONS

This application claims the benefit of U. S. Provisional Application 60/739,117, filed Nov. 23, 2005, and U. S. Provisional Application 60/751,962, filed Dec. 20, 2005. The contents of each of the aforementioned applications is hereby incorporated by reference herein.

### GOVERNMENT SUPPORT

The following invention was supported at least in part by from Department of Defense Grant OC0400600 and NIH grant CA103937. Accordingly, the government may have certain rights in the invention.

### BACKGROUND OF THE INVENTION

Currently there are more than 1.3 million people that are diagnosed with cancer each year in the United States alone. Additionally, nearly 500,000 people die from cancer in the United States each year.

The identification of tumor markers suitable for the early detection and diagnosis of cancer holds great promise to improve the clinical outcome of patients. It is especially important for patients presenting with vague or no symptoms or with tumors that are relatively inaccessible to physical examination. Ovarian carcinoma represents one of such insidious and aggressive cancers. It is the most lethal gynecologic malignancy in women with approximately 25,000 new cases in the United States each year. E. Banks et al. Int. J Gynecol Center (1997) 7:425-38; D. M. Parkin etal., IARC Scientif (1992); R. T. Greenlee etal., CA Cancer J. Clin (2001) 51:15-37. Despite considerable effort directed at early detection, no cost effective screening tests have been developed and women generally present with disseminated disease at diagnosis. P. J. Paley, Curr Opin Oncol, (2001) 13(5); R. F. Ozols et al., Principles and Practice of Gyneologic Oncology, 3^{rd} ed. Philadelphia: Lippincott, Williams and Wilkins, 2000, pp.: 981-1057.

Epithelial ovarian carcinoma is the most common and most lethal of all gynecologic malignancies. Only 30% of ovarian tumors are diagnosed at an early stage (Stage I/II), when survival rates reach 90%. The rest are diagnosed at an advanced stage, with survival rates of less than 20% (Greenlee R T, Hill-Harmon M B, Murray T, et al., 2001. CA Cancer J Clin.2001;51:15-36).

Accordingly, the need exists for the identification of novel cancer biomarkers that will allow for detection of cancer, e.g., ovarian cancer. Moreover, the need exists for additional therapeutic targets for the treatment and prevention of cancer.

### SUMMARY OF THE INVENTION

The instant application is based, at least in part, on the discovery that the nucleic acid encoding RSF-1 and the RSF-1 polypeptide are overexpressed in cancerous tissue. Moreover, the level of RSF-1 polypeptide and nucleic acid expression is correlated with the prognosis of a subject having cancer.

Accordingly, in one aspect, the invention provides methods for detecting cancer in a subject by detecting the amount of the nucleic acid of SEQ ID NO:1, or a fragment thereof, in a biological sample from the subject, wherein the overexpression of SEQ ID NO:1 is indicative that the subject has cancer. In one embodiment, the amount of the nucleic acid of SEQ ID NO:1 or a fragment thereof, is detected by FISH.

In another embodiment, the biological sample is from a solid tumor, e.g., a liver, breast, lung, or ovarian tumor.

In another embodiment, the sample is an ovarian tissue sample.

In another aspect, the invention provides methods for detecting cancer in a subject by detecting the amount of the polypeptide of SEQ ID NO:2, or a fragment thereof, in a biological sample from the subject, wherein the overexpression of SEQ ID NO:2 is indicative that the subject has cancer.

In one embodiment, the polypeptide of SEQ ID NO:2 or a fragment thereof, is detected by immunohistochemistry.

In another embodiment, the biological sample is from a solid tumor, e.g., a liver, breast, lung, or ovarian tumor.

In another embodiment, the sample is an ovarian tissue sample.

In another aspect, the invention provides methods for determining the aggressiveness of cancer in a subject, by detecting the amount of SEQ ID NO:1 in a biological sample from the subject, wherein the level of overexpression of SEQ ID NO:1 as compared to a control is indicative of the aggressiveness of cancer.

In one embodiment, the amount of the nucleic acid of SEQ ID NO:1 or a fragment thereof, is detected by FISH.

In another embodiment, the biological sample is from a solid tumor, e.g., a liver, breast, lung, or ovarian tumor.

In another embodiment, the sample is an ovarian tissue sample.

In another embodiment, the method further comprises the step of treating the subject based on the aggressiveness of the cancer.

In another aspect, the invention provides methods for determining the aggressiveness of cancer in a subject, by detecting the amount of SEQ ID NO:2 in a biological sample from the subject, wherein an overexpression of SEQ ID NO:2 as compared to a control is indicative of the aggressiveness of cancer.

In one embodiment, the polypeptide of SEQ ID NO:2 or a fragment thereof, is detected by immunohistochemistry.

In another embodiment, the biological sample is from a solid tumor, e.g., a liver, breast, lung, or ovarian tumor.

In another embodiment, the sample is an ovarian tissue sample.

In another embodiment, the method further comprises the step of treating the subject based on the aggressiveness of the cancer.

In another aspect, the invention provides, methods of determining the aggressiveness of ovarian cancer in a subject by determining the amount of the polypeptide of SEQ ID NO:2 present in a biological sample from the subject, wherein an amount higher than a control level indicates that the subject has aggressive ovarian cancer.

In another aspect, the invention provides methods for determining the prognosis of a subject with cancer, e.g., ovarian cancer, by determining the amount of the nucleic acid of SEQ ID NO:1 in a biological sample, wherein the prognosis of an individual is related to the amount of the nucleic acid of SEQ ID NO:1 in a sample. In a related embodiment, the higher the amount of the nucleic acid of SEQ ID NO:1 in a sample, the worse the prognosis for the subject.

In one embodiment, the amount of the nucleic acid of SEQ ID NO:1 or a fragment thereof, is detected by FISH.

In another embodiment, the biological sample is from a solid tumor, e.g., a liver, breast, lung, or ovarian tumor.

In another embodiment, the sample is an ovarian tissue sample.

In another embodiment, the method further comprises the step of treating the subject based on the aggressiveness of the cancer.

In another aspect, the invention provides methods for determining the prognosis of an subject having cancer, by detecting the amount of the polypeptide of SEQ ID NO:2, or a fragment thereof, in a biological sample from the subject, wherein the prognosis of an individual is related to the amount of the polypeptide of SEQ ID NO:2 in a sample. In a related embodiment, the higher the amount of the polypeptide of SEQ ID NO:2 in a sample, the worse the prognosis for the subject.

In one embodiment, the polypeptide of SEQ ID NO:2 or a fragment thereof, is detected by immunohistochemistry.

In another embodiment, the biological sample is from a solid tumor, e.g., a liver, breast, lung, or ovarian tumor.

In another embodiment, the sample is an ovarian tissue sample.

In another embodiment, the method further comprises the step of treating the subject based on the aggressiveness of the cancer.

In another aspect, the invention provides, methods of determining the prognosis of a subject with cancer, e.g., ovarian cancer, by determining the amount of the polypeptide of SEQ ID NO:2 present in a biological sample from the subject, wherein an amount higher than a control level is indicative of poor prognosis.

In a related embodiment, the methods further comprise the step of treating a subject based on the prognosis.

In another aspect, the invention provides a method of determining the prognosis of a subject having ovarian cancer by determining the amount of the polypeptide of SEQ ID NO:2 in a biological sample from the subject, wherein the level SEQ ID NO:2 as compared to one or more control levels is indicative of the prognosis of the subject.

In another aspect, the invention provides methods for identifying a compound for treating or preventing of cancer comprising, contacting a cell with a test compound, determining if the test compound inhibits the expression of the nucleic acid of SEQ ID NO:1, thereby identifying a compound for the treatment or prevention of cancer.

In another aspect, the invention provides methods for identifying a compound for the treatment or prevention of cancer comprising, contacting a cell with a test compound, determining if the test compound inhibits the expression or activity of the polypeptide of SEQ ID NO:2, thereby identifying a compound for the treatment or prevention of cancer.

In related embodiments, the compound is a small molecule, a peptide, a polypeptide, or a nucleic acid molecule. In another related embodiment, the peptide or polypeptide is an antibody or fragment thereof, the nucleic acid molecule is an siRNA, shRNA, antisense nucleic acid, or ribozyme.

In another embodiment, the cancer is selected from the group consisting of ovarian, lung, liver and breast cancer. In a specific embodiment, the cancer is ovarian cancer.

In another embodiment, the invention provides methods of treating or preventing cancer in a subject by administering to a subject a compound that inhibits the expression or activity of a nucleic acid of SEQ ID NO:1 or a polypeptide of SEQ ID NO:2, thereby treating the subject.

In another embodiment, the cancer is selected from the group consisting of ovarian, lung, liver and breast cancer. In a specific embodiment, the cancer is ovarian cancer.

In one embodiment, the compound is selected from the group consisting of a small molecule, a peptide, a polypeptide, and a nucleic acid molecule. In a related embodiment, the peptide or polypeptide is an antibody or fragment thereof. In another related embodiment, the nucleic acid molecule is an siRNA, shRNA, antisense nucleic acid, or ribozyme.

In another aspect, the invention provides kits comprising an antibody, or fragment thereof, for use in determining the amount of the polypeptide of SEQ ID NO:2 in a sample and instructions for use. In one embodiment, the kit further comprises a control. In another embodiment, the kit is for the diagnosis of cancer. In a related embodiment, the kit is for determining the aggressiveness of cancer, e.g., ovarian, lung, liver or breast cancer.

In another aspect, the invention provides kits comprising a nucleic acid probe for use in determining the amount of the nucleic acid of SEQ ID NO:1 in a sample and instructions for use. In one embodiment, the kit further comprises a control. In another embodiment, the kit is for the diagnosis of cancer. In a related embodiment, the kit is for determining the aggressiveness of cancer, e.g., ovarian, lung, liver or breast cancer. In another embodiment, the probe comprises a label.

### DESCRIPTION OF THE DRAWINGS

FIGS. 1A-C depict the identification of the 11q13.5 amplicon in ovarian cancers. A) Digital karyotyping identified 3 ovarian carcinomas that contained discrete amplifications at 11q13.5 region. Alignment of the amplicons revealed a common region of amplification (blue line at the bottom panel) spanning from 76.6 Mb to 78.4 Mb at chromosome 11q. Red and green arrows indicate the physical locations of EMSY and Rsf-1, respectively. B) Validation of 11q13.5 amplification was performed by FISH analysis in the same 3 tumors using a probe (green) located within the minimal amplicon of 11q13.5 and a control probe (red) located at 11q11 (21 Mb centromeric to the minimal amplicon). C) 11q13.5 amplification was further validated in the three tumors using quantitative real-time PCR on genomic DNA. For each tumor, an increase in the DNA copy number (y-axis) is present at a specific sub-chromosomal region which corresponds to the amplification identified by digital karyotyping. The dash line indicates a cutoff of 2.2 which represents the threshold for amplification with confidence level of 97.5%.

FIG. 2 depicts gene expression analysis of the 11q13.5 amplicon in ovarian tumors. Quantitative real-time PCR was performed for all 13 genes located within the minimal amplicon in benign cystadenomas, low-grade ovarian carcinomas and high-grade ovarian carcinomas with or without 11q13.5 amplification. The expression level of each gene (left to right: centromeric to telomeric) in individual specimen is shown as a pseudo-color gradient based on the relative expression level of a given specimen to the normal ovarian surface epithelium. Right panel indicates the amplification status of Rsf-1, EMSY and the 11q11 (control locus for FISH) for each specimen that was determined by FISH analysis. Filled circles indicate amplification and open circles indicate no amplification.

FIGS. 3A-B depict correlation of Rsf-1 DNA copy numbers and Rsf-1 protein expression in high-grade ovarian carcinomas. A) The specificity of the anti-Rsf-1 antibody is demonstrated by Western blot analysis. OSE: ovarian surface epithelial cells; 293: human embryonic kidney (HEK) 293 cells; 293T: HEK293 cells transfected with a full-length Rsf-1 gene. A predominant band of Rsf-1 protein at 215 kD that represents the full-length Rsf-1 gene is detected in 293T cells. The faint lower band represents the degradation product of Rsf-1 protein. Endogenous Rsf-1 expression is also observed in 293 cells but not in OSE cells. Bottom panel shows the GAPDH expression as the loading control. B) Left: A high-grade carcinoma shows weak RSF immunoreactivity (1+) and does not display Rsf-1 gene amplification. Right: A high-grade tumor demonstrates an intense Rsf-1 immunoreactivity (4+) and displays Rsf-1 gene amplification. c. Rsf-1 protein expression correlates with the Rsf-1 gene copy number in high-grade ovarian carcinomas. Tumors with the highest copy number of Rsf-1 DNA (manifested as homogenous staining regions, HSR) express the highest level of Rsf-1 protein (4+). Tumors that lack Rsf-1 amplification demonstrate weak to moderate Rsf-1 immunoreactivity (1+and 2+). Each dot represents an individual specimen. Among 16 amplified tumors, there are 15 available for immunohistochemistry.

FIGS. 4A-B demonstrate that Rsf-1 amplification and over-expression correlate with shorter overall survival in patients. A) Kaplan-Meier survival analysis shows that Rsf-1 amplification (solid line, n=16) is associated with a shorter overall survival compared to tumors without Rsf-1 amplification (dash line, n=91) (p=0.03, Log-rank test). B) Quantitative real-time PCR in effusion samples of ovarian high-grade serous carcinomas demonstrates that Rsf-1 over-expression (>2.4 fold of normal ovarian surface epithelium; solid line; n=11) correlates significantly with shorter overall survival than those with a low expression level (<2.4 fold; dash line; n=42) (p=0.042, Log-rank test).

FIGS. 5A-E depict the functional analyses of Rsf-1 expression. A) Western blot analysis shows that Rsf-1 transfected RK3E clones (C-1,-2 and -3) express Rsf-1 protein with a predominant molecular mass of 215 kD which is similar to the endogenous Rsf-1 protein expressed in OVCAR3 cells. Control RK3E cells, which are transfected with an empty vector, do not express Rsf-1 protein. As compared to the control RK3E cells, Rsf-1 clones continue proliferating at low serum concentrations (0.5% and 0.2%). B) The Rsf-1 clones demonstrate a higher proliferative activity than the vector only control as evidenced by a time-dependent increase in cell number at low (0.5%) serum-containing medium. C) Anchorage-independent assay demonstrates that colonies observed in the Rsf-1 clones are more than those in the vector only control. D) Effects of Rsf-1 gene knockdown. Knockdown of Rsf-1 significantly reduces cell number in OVCAR3 cells which harbor Rsf-1 amplification and in HeLa cells that express Rsf-1. In contrast, Rsf-1 siRNA has only a minimal effect on cell growth in ovarian surface epithelial cells (OSE) which do not express detectable Rsf-1. E) Rsf-1 targeting siRNA reduces cell proliferation as measured by the percentage of BrdU positive cells but not in control siRNA or in non-treated (mock control) OVCAR3 cells. The percentage of apoptotic cells as measured by annexin V staining is similar among Rsf-1 siRNA, control siRNA and non-treated OVCAR3 cells.

FIG. 6 depicts digital karyotyping view of chromosome 11. Seven digital karyotyping libraries were generated from 6 ovarian serous tumors and on ovarian cancer cell line (OVCAR3). Amplification at 11q13.5 is identified in 3 libraries (Tumor 1, Tumor 2 and OVCAR3). No evidence of other amplicons is detected in all libraries.

FIG. 7 demonstrates the physical locations of genes within the minimal amplicon. The locations of 13 genes within the minimal amplicon on chromosome 11 are indicated by pink bars. Minimal amplicon delineated by digital karyotyping is shown as a blue bar and the FISH probe that hybridizes to the epicenter of the minimal amplicon is shown as a green bar.

FIG. 8 depict the dual-color FISH analysis of Rsf-1 and EMSY genes in ovarian carcinomas. Upper panel: Tumor 1 used in digital karyotyping (FIG. 6a) shows an HSR pattern of Rsf-1 amplification (green) but lacks EMSY amplification (red). Bottom panel: another tumor exhibits Rsf-1 amplification without evidence of EMSY gene amplification.

FIG. 9 depict Rsf-1 knockdown by siRNA. Rsf-1 siRNA significantly reduces the mRNA levels of Rsf-1 in both OVCAR3 and HeLa cells as compared to control (scramble) siRNA treated cells. The mRNA expression level is normalized to OSE (ovarian surface epithelial cells) and is expressed as a fold change.

FIGS. 10A-C depict the effects of Rsf-1 gene knockdown in a mouse xenograft model. A: Western blot analysis demonstrates a remarkable reduction in Rsf-1 protein expression in OVCAR3 cells transfected with Rsf-1 shRNA as compared to the cells transfected with control (scramble) sequence shRNA. B: Rsf-1 shRNA transfected cells grow very small tumor nodules in the peritoneal cavity. In contrast, the control shRNA transfected cells grow much larger intraperitoneal tumors. Tumor were excised and weighted. C. Control shRNA transfected OVCAR3 cells grow tumor in a representative mouse (left panel) while Rsf-1 shRNA transfected cells do not grow obvious tumors in a representative mouse (right panel).

FIGS. 11A-B set froth the nucleic acid and polypeptide sequence of RSF-1 as SEQ ID NO:1 and SEQ ID NO:2, respectively.

FIGS. 12A-E demonstrate that Rsf-1 protein expression is frequently expressed in ovarian carcinoma cells in effusions: A-B: Two effusions (A peritoneal, B pleural) showing strong nuclear expression. Reactive cells, mainly lymphocytes, are negative. C-D: Two effusions (C pleural, D peritoneal) with weak expression. E: Rsf-1-negative peritoneal specimen.

FIGS. 13A-E demonstrate that Rsf-1 expression correlates with shorter overall survival (OS) in patients with post-chemotherapy effusions. A: Kaplan-Meier survival curve showing the trend for correlation between higher Rsf-1 staining score (extent×intensity, see materials and methods) and poor OS for the entire cohort (=135 patients). Patients with effusions with high (>4) Rsf-1 expression score (=68, dashed line) had a mean OS of 27 months (median=24 months) compared to 35 months (median=29 months) for patients with tumors with low (≦4) expression score (=67, solid line; p=0.081). B: Kaplan-Meier survival curve showing the correlation between Rsf-1 expression score and OS for patients with post-chemotherapy effusions. Patients with effusions showing high (>4) Rsf-1 expression score (=25, dashed line) had a mean OS of 29 months (median=25 months) compared to 42 months (median=38 months) for patients with tumors with low (<4) expression score (=34, solid line; p=0.02). C: Kaplan-Meier survival curve showing the correlation between Rsf-1 expression extent (percentage of stained cells) and OS. Patients with effusions with Rsf-1 expression in 76%-100 of cells (=24, dashed line) had a mean OS of 28 months (median=24 months) compared to 42 months (median=38 months) for patients with tumors with more focal expression (=35, solid line; p=0.009). D: Kaplan-Meier survival curve showing the correlation between Rsf-1 expression intensity and OS. Patients with tumor cells showing strong Rsf-1 expression (=27, dashed line) had a mean OS of 29 months (median=25 months) compared to 43 months (median=40 months) for patients with tumors with more focal expression (=32, solid line; p=0.009). E: Kaplan-Meier survival curve showing the correlation between FIGO stage and OS. Patients with effusions with FIGO stage IV disease (=14, dashed line) had a mean OS of 26 months (median=20 months) compared to 39 months (median=32 months) for patients with stage III disease (=43, solid line; p=0.032).

FIG. 14 sets forth Table 2: Clinicopathologic data of the study cohort (135 patients) from Example 2.

FIGS. 15A-D depict the results of a multi location survival study. Overall survival length was plotted against the level of RSF-1 polypeptide or nucleic acid expression.

### DETAILED DESCRIPTION OF THE INVENTION

The instant invention is based, at least in part, on the discovery by the inventors of the instant application that the presence and level of RSF-1 corresponds to the presence of cancer and the prognosis of an individual with cancer.

As used herein, the term “cell-proliferative disorder” denotes malignant as well as nonmalignant populations of transformed cells which morphologically often appear to differ from the surrounding tissue. In one embodiment, the cell proliferative disorder is cancer.

As used herein, “prognosis” refers to a subjects prospect of survival and recovery from cancer as anticipated from the usual course of the cancer. In one embodiment, the prognosis of a subject can be determined by determining the amount of RSF polypeptide or nucleic acid in a sample from the subject and comparing that amount to the amount present in individuals who have had similar cancers and whose fait is known. In various aspects, the aggressiveness of therapy is based on the prognosis of the subject.

As used herein, “transformed cells” refers to cell which have spontaneously converted to a state of unrestrained growth, i.e., they have acquired the ability to grow through an indefinite number of divisions in culture. Transformed cells may be characterized by such terms as neoplastic, anaplastic and/or hyperplastic with respect to their loss of growth control.

As used herein, the term “cancer” is used to mean a condition in which a cell in a subject's body undergoes abnormal, uncontrolled proliferation. Thus, “cancer” is a cell-proliferative disorder. Non-limiting examples of cancers include breast cancer, ovarian cancer, cervical cancer, prostate cancer, colon cancer, lung cancer, skin cancer, leukemia, lymphoma, melanoma or any other type of cancer.

“Administering” is defined herein as a means providing the composition to the subject in a manner that results in the composition being inside the subject's body. Such an administration can be by any route including, without limitation, subcutaneous, intradermal, intravenous, intra-arterial, intraperitoneal, and intramuscular.

By “treating” a subject or subjecting a subject to “treatment”, it is meant that the subject's symptoms are partially or totally alleviated, or remain static following treatment according to the invention. A subject that has been treated can exhibit a partial or total alleviation of symptoms (for example, tumor load). The term “treatment” is intended to encompass prophylaxis, therapy and cure.

A “therapeutically effective amount” is defined herein an effective amount of composition for producing some desire therapeutic effect.

A physician or veterinarian having ordinary skill in the art can readily determine and prescribe the “effective amount” (ED₅₀) of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

The term “sample” is defined herein as blood, blood product, biopsy tissue, serum, and any other type of fluid or tissue that can be extracted from a subject or a mammal. The terms “sample” and “biological sample” are used interchangeably in this application.

As used herein, a “subject” can be any mammal.

RSF-1 polypeptides, or nucleic acids encoding RSF-1 polypeptides, or portions thereof may act as markers useful in the detection of a cell-proliferative disorder, the monitoring of a cell-proliferative disorder or as targets for treating a cell-proliferative disorder.

As used herein a “RSF-1 marker” refers to a RSF-1 polypeptide or a nucleic acid (such as an mRNA) encoding a RSF-1 polypeptide.

As used herein the term “RSF-1 polypeptide” refers to the full-length RSF-1 polypeptide, or fragment thereof. Thus, the term “RSF-1 polypeptide” includes fragments of RSF-1.

In one embodiment, the RSF-1 polypeptide of the invention is encoded by SEQ ID NO:1, or a fragment thereof.

In another embodiment, the RSF-1 polypeptide of the invention is encoded by a nucleic acid that hybridizes to SEQ ID NO:1 under stringent conditions.

In another embodiment, the RSF-1 polypeptide comprises the amino acid sequence of SEQ ID NO:2, or a fragment thereof.

In another embodiment, the RSF-1 polypeptide comprises an amino acid sequence having conservative amino acid substitutions as compared to SEQ ID NO:2, or a fragment of said amino acid sequence.

Variants of RSF-1

The claimed invention includes the use of variants of the RSF-1 polypeptides. Variants of the present invention may have an amino acid sequence that is different by one or more amino acid substitutions to the amino acid sequence disclosed in SEQ ID NO:2. Embodiments which comprise amino acid deletions and/or additions are also contemplated. The variant may have conservative changes (amino acid similarity), wherein a substituted amino acid has similar structural or chemical properties, for example, the replacement of leucine with isoleucine. Guidance in determining which and how many amino acid residues may be substituted, inserted, or deleted without abolishing biological or proposed pharmacological activity may be reasonably inferred in view of this disclosure and may further be found using computer programs well known in the art, for example, DNAStar™ software.

Amino acid substitutions may be made, for instance, on the basis of similarity in polarity, charge, solubility, hydrophobicity, hydrophilicity, and/or the amphipathic nature of the residues as long as a biological and/or pharmacological activity of the native molecule is retained.

Negatively charged amino acids include aspartic acid and glutamic acid; positively charged amino acids include lysine and arginine; amino acids with uncharged polar head groups having similar hydrophilicity values include leucine, isoleucine, and valine; amino acids with aliphatic head groups include glycine, alanine; asparagine, glutamine, serine; and amino acids with aromatic side chains include tryptophan, phenylalanine, and tyrosine.

“Identity” is a measure of the similarity of nucleotide sequences or amino acid sequences. In order to characterize the identity, subject sequences are aligned so that the highest percentage identity (match) is obtained, after introducing gaps, if necessary, to achieve maximum percent identity. N- or C-terminal extensions shall not be construed as affecting identity. “Identity” per se has an art-recognized meaning and can be calculated using published techniques. Computer program methods to determine identity between two sequences, for example, include DNAStar™ software (DNAStar Inc. Madison, Wis.); the GCG™ program package (Devereux, J., et al. Nucleic Acids Research (1984) 12(1): 387); BLASTP, BLASTN, FASTA (Atschul, S. F. et al., J. Molec Biol (1990) 215: 403). Homology (identity) as defined herein is determined conventionally using the well-known computer program, BESTFIT™ (Wisconsin Sequence Analysis Package, Version 8 for Unix, Genetics Computer Group, University Research Park, 575 Science Drive, Madison, Wis., 53711). When using BESTFIT™ or any other sequence alignment program (such as the Clustal algorithm from MegAlign software (DNAStar™) to determine whether a particular sequence is, for example, about 90% homologous to a reference sequence, according to the present invention, the parameters are set such that the percentage of identity is calculated over the full length of the reference nucleotide sequence or amino acid sequence and that gaps in homology of up to about 90% of the total number of nucleotides in the reference sequence are allowed.

In one embodiment, the RSF-1 polypeptide is a variant of SEQ ID NO:2. In one embodiment, the RSF-1 polypeptide is at least 99%, 98%, 97%, 96%, 95%, 90%, 85% or 80% identical to SEQ ID NO:2. In another embodiment, the RSF-1 polypeptide is encoded by a nucleic acid that is at least 99%, 98%, 97%, 96%, 95%, 90%, 85% or 80% homologous to SEQ ID NO:1.

RSF-1 Modulators

The present invention also provides RSF-1 modulators. In one embodiment, the RSF-1 modulators are RSF-1 antagonists. As used herein, a “RSF-1 antagonist” is any molecule which inhibits the biological or functional effect of naturally occurring RSF-1. A RSF-1 antagonist may inhibit the biological or functional effect of naturally occurring RSF-1 by any means.

In one embodiment, a RSF-1 antagonist inhibits the biological or functional effect of naturally occurring RSF-1 by decreasing the expression or activity of RSF-1. In another embodiment, a RSF-1 antagonist inhibits the biological or functional effect of naturally occurring RSF-1 by specifically binding to RSF-1.

A RSF-1 antagonist may be a peptide or a peptidomimetic, an antibody or fragment thereof that binds RSF-1, a nucleic acid molecule, or a small molecule.

Antibodies to RSF-1

Another aspect of the invention pertains to antibodies, or fragments thereof, which specifically binds to a RSF-1.

In one embodiment, the invention comprises an isolated antibody or fragment thereof which binds specifically to a RSF-1 polypeptide. In one embodiment, the antibody or fragment thereof binds specifically to a RSF-1 polypeptide encoded by a nucleic acid comprising SEQ ID NO:1, or comprising the amino acid sequence of SEQ ID NO:2. In another embodiment, the antibody or fragment thereof binds specifically to the extracellular domain of a RSF-1 polypeptide.

In one embodiment, the invention comprises an isolated antibody or fragment thereof which is a RSF-1 antagonist.

In one embodiment, the antibody or fragment thereof further comprises a label, wherein the label is selected from the group consisting of a fluorescent label, a radiolabel, a toxin, a metal compound and biotin. In one embodiment the fluorescent label is selected from the group consisting of Texas Red, phycoerythrin (PE), cytochrome c, and fluorescent isothiocyante (FITC). In another embodiment, the radiolabel is selected from the group consisting of ³²p, ³³p,⁴³K, ⁴⁷Sc, ⁵²Fe, ⁵⁷Co, ⁶⁴Cu, ⁶⁷Ga, ⁶⁷Cu, ⁶⁸Ga, ⁷¹Ge, ⁷⁵Br, ⁷⁶Br, ⁷⁷Br, ⁷⁷As, ⁷⁷Br, ⁸¹Rb/⁸¹MKr, ⁸⁷MSr, ⁹⁰Y, ⁹⁷Ru, ⁹⁹Tc, ¹⁰⁰Pd, ¹⁰¹Rh, ¹⁰³Pb, ¹⁰⁵Rh, ¹⁰⁹Pd, ¹¹¹Ag, ¹¹¹In, ¹¹³In, ¹¹⁹Sb, ¹²¹Sn, ¹²³I, ¹²⁵I, ¹²⁷Cs, ¹²⁸Ba, ¹²⁹Cs, ¹³¹I, ¹³¹Cs, ¹⁴³Pr, ¹⁵³Sm, ¹⁶¹Tb, ¹⁶⁶Ho, ¹⁶⁹Eu, ¹⁷⁷Lu, ¹⁸⁶Re, ¹⁸⁸Re, ¹⁸⁹Re, ¹⁹¹Os, ¹⁹³Pt, ¹⁹⁴Ir, ¹⁹⁷Hg, ¹⁹⁹Au, ²⁰³Pb, ²¹¹At, ²¹²Pb, ²¹²Bi and ²¹³Bi. In another embodiment, the toxin is selected from the group consisting of ricin, ricin A chain (ricin toxin), Pseudomonas exotoxin (PE), diphtheria toxin (DT), Clostridium perfringens phospholipase C (PLC), bovine pancreatic ribonuclease (BPR), pokeweed antiviral protein (PAP), abrin, abrin A chain (abrin toxin), cobra venom factor (CVF), gelonin (GEL), saporin (SAP), modeccin, viscumin and volkensin.

A person of skilled in the art would know how to make antibodies or fragments thereof which specifically bind to a RSF-1. For example, by using peptides based on the sequence of the subject proteins, specific antisera or monoclonal antibodies can be made using standard methods. Chickens, or a mammal such as a mouse, a hamster or rabbit can be immunized with an immunogenic form of the peptide (e.g., an antigenic fragment which is capable of eliciting an antibody response). Techniques for conferring immunogenicity on a protein or peptide include conjugation to carriers or other techniques well known in the art. For instance, a peptidyl portion of one of the subject proteins can be administered in the presence of adjuvant. The progress of immunization can be monitored by detection of antibody titers in plasma or serum. Standard ELISA or other immunoassays can be used with the immunogen as antigen to assess the levels of antibodies.

Following immunization, antisera can be obtained and, if desired, polyclonal antibodies against the target protein can be further isolated from the serum. To produce monoclonal antibodies, antibody producing cells (lymphocytes) can be harvested from an immunized animal and fused by standard somatic cell fusion procedures with immortalizing cells such as myeloma cells to yield hybridoma cells. Such techniques are well known in the art, and include, for example, the hybridoma technique (originally developed by Kohler and Milstein, Nature, 256: 495497, 1975), as well as the human B cell hybridoma technique (Kozbar et al., Immunology Today, 4: 72, 1983), and the EBV-hybridoma technique to produce human monoclonal antibodies (Cole et al., Monoclonal Antibodies and Cancer Therapy, Alan R. Liss, Inc. pp. 77-96, 1985). Hybridoma cells can be screened immunochemically for production of antibodies specifically reactive with the RSF-1 polypeptides and the monoclonal antibodies isolated.

The term antibody as used herein is intended to include fragments thereof which are also specifically reactive with one of the subject proteins or complexes including the subject proteins. Antibodies can be fragmented using conventional techniques and the fragments screened for utility in the same manner as described above for whole antibodies. For example, F(ab′)2 fragments can be generated by treating antibody with pepsin. The resulting F(ab′)2 fragment can be treated to reduce disulfide bridges to produce Fab′ fragments. The antibody of the present invention is further intended to include bispecific and chimeric molecules, as well as single chain (scFv) antibodies.

The subject antibodies include humanized antibodies, which can be prepared as described, e.g., in U.S. Pat. No. 5,585,089. Also within the scope of the invention are single chain antibodies. All of these modified forms of antibodies as well as fragments of antibodies are intended to be included in the term “antibody” and are included in the broader term “binding moiety”.

Antibodies of the present invention can be made recombinantly. Linkers may be added to the nucleic acid sequences of the heavy and light chains to increase flexibility of the antibody. In the case of a scFv, the linkers are added to connect the VH and VL chains and the varying composition can effect solubility, proteolytic stability, flexibility, and folding.

Peptides and Peptidomimetics

One embodiment of the present inventions are peptides, and compositions thereof, which may be used to detect a RSF-1 polypeptide. Peptides of the present invention can comprise 5-50 amino acid residues. More preferably, peptides of the present invention comprise 5-30 amino acid residues. More preferably, peptides of the present invention comprise 5-20 amino acid residues. More preferably, peptides of the present invention comprise 10-15 amino acid residues.

Another aspect of the invention provides a peptide or peptidomimetic, e.g., wherein one or more backbone bonds are replaced or one or more side chains of a naturally occurring amino acid are replaced with sterically and/or electronically similar functional groups.

In certain embodiments, the peptide or peptidomimetic is formulated in a pharmaceutically acceptable excipient.

Pharmaceutical Compositions

Each of the embodiments of the present invention can be used as a composition when combined with a pharmaceutically acceptable carrier or excipient. Pharmaceutically acceptable carriers are physiologically acceptable and retain the therapeutic properties of the antibodies or peptides present in the composition. Pharmaceutically-acceptable carriers are well-known and generally described in, for example, Remington's Pharmaceutical Sciences (18.sup.th Edition, ed. A. Gennaro, Mack Publishing Co., Easton, Pa., 1990). On exemplary pharmaceutically acceptable carrier is physiological saline.

Chemotherapeutic Agents

The instant invention also provides methods of treating a subject using a combination treatment. In one example, a RSF-1 modulator is used with a chemotherapeutic agent. Chemotherapeutic agents contemplated by the present invention include chemotherapeutic drugs that are commercially available.

Merely to illustrate, the chemotherapeutic can be an inhibitor of chromatin function, a topoisomerase inhibitor, a microtubule inhibiting drug, a DNA damaging agent, an antimetabolite (such as folate antagonists, pyrimidine analogs, purine analogs, and sugar-modified analogs), a DNA synthesis inhibitor, a DNA interactive agent (such as an intercalating agent), and/or a DNA repair inhibitor.

Chemotherapeutic agents may be categorized by their mechanism of action into, for example, the following groups: anti-metabolites/anti-cancer agents, such as pyrimidine analogs (5-fluorouracil, floxuridine, capecitabine, gemcitabine and cytarabine) and purine analogs, folate antagonists and related inhibitors (mercaptopurine, thioguanine, pentostatin and 2-chlorodeoxyadenosine (cladribine)); antiproliferative/antimitotic agents including natural products such as vinca alkaloids (vinblastine, vincristine, and vinorelbine), microtubule disruptors such as taxane (paclitaxel, docetaxel), vincristin, vinblastin, nocodazole, epothilones and navelbine, epidipodophyllotoxins (etoposide, teniposide), DNA damaging agents (actinomycin, amsacrine, anthracyclines, bleomycin, busulfan, camptothecin, carboplatin, chlorambucil, cisplatin, cyclophosphamide, cytoxan, dactinomycin, daunorubicin, doxorubicin, epirubicin, hexamethylmelamineoxaliplatin, iphosphamide, melphalan, merchlorehtamine, mitomycin, mitoxantrone, nitrosourea, plicamycin, procarbazine, taxol, taxotere, teniposide, triethylenethiophosphoramide and etoposide (VP16)); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes-dacarbazinine (DTIC); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory agents; antisecretory agents (breveldin); immunosuppressives (cyclosporine, tacrolimus (FK-506), sirolimus (rapamycin), azathioprine, mycophenolate mofetil); anti-angiogenic compounds (TNP470, genistein) and growth factor inhibitors (vascular endothelial growth factor (VEGF) inhibitors, fibroblast growth factor (FGF) inhibitors); angiotensin receptor blocker; nitric oxide donors; anti-sense oligonucleotides; antibodies (trastuzumab, rituximab); cell cycle inhibitors and differentiation inducers (tretinoin); mTOR inhibitors, topoisomerase inhibitors (doxorubicin (adriamycin), amsacrine, camptothecin, daunorubicin, dactinomycin, eniposide, epirubicin, etoposide, idarubicin, irinotecan (CPT-11) and mitoxantrone, topotecan, irinotecan), corticosteroids (cortisone, dexamethasone, hydrocortisone, methylpednisolone, prednisone, and prenisolone); growth factor signal transduction kinase inhibitors; mitochondrial dysfunction inducers, toxins such as Cholera toxin, ricin, Pseudomonas exotoxin, Bordetella pertussis adenylate cyclase toxin, or diphtheria toxin, and caspase activators; and chromatin disruptors. Preferred dosages of the chemotherapeutic agents are consistent with currently prescribed dosages.

Methods of Diagnosis

The invention provides a method for diagnosis of a cell-proliferative disorder in a subject comprising detecting the presence of a RSF-1 marker in a sample, wherein the presence of said marker is indicative of the cell-proliferative disorder. In one embodiment, the cell-proliferative disorder is cancer. In another embodiment, the cancer is breast cancer, ovarian, cervical cancer, prostate cancer, colon cancer, lung cancer, skin cancer, leukemia, lymphoma, melanoma or any other type of cancer. In one embodiment the cancer is ovarian cancer.

The invention further provides methods of diagnosing cancer in a subject by determining the level of RSF-1 in combination with one or more additional cancer markers. In a specific example, RSF-1 levels are measured along with one or more additional ovarian cancer markers for the detection of ovarian cancer.

The invention also provides a method for assessing RSF-1 prognosis of a subject comprising detecting the presence and/or amount of a RSF-1 marker in a biological sample obtained from a subject. In one embodiment, the method for assessing RSF-1 presence further comprising quantifying the amount of RSF-1 marker in the biological sample, wherein the amount of RSF-1 marker in the biological sample is indicative the prognosis or a subject.

The invention also provides methods for determining the aggressiveness of cancer in a subject by detecting the presence or amount of a RSF-1 marker in an biological sample.

The RSF-1 marker can be any of the markers described above. In one embodiment, the RSF-1 marker is a RSF-1 polypeptide or a fragment thereof. In a another embodiment, the marker is a RSF-1 nucleic acid.

In one embodiment the RSF-1 marker is a RSF-1 polypeptide encoded by a nucleic acid comprising SEQ ID NO:1 or a fragment thereof. In another one embodiment the RSF-1 marker is a polypeptide encoded by a nucleic acid that hybridizes to SEQ ID NO:1 under stringent conditions.

In another embodiment, the RSF-1 marker is a RSF-1 polypeptide which comprises the amino acid sequence of SEQ ID NO:2 or a fragment thereof.

The methods of the present invention may be performed in any relevant sample. A sample can be a tissue, a cell or a body fluid. In one embodiment, the tissue is ovarian tissue, preferably biopsy tissue. The body fluid can be any body fluid, including but not limited to blood, serum, plasma, urine, saliva, sputum and breast ductal secretions. In one embodiment, the body fluid is blood or serum.

Protein Based Assays

In one embodiment, the RSF-1 marker is a RSF-1 polypeptide or a fragment thereof. A RSF-1 polypeptide may be detected using any assay method available in the art, a subset of which is discussed below. Non-limiting examples of such methods include immunohistochemistry, ELISAs, MRI and Western blots.

In one embodiment the presence of RSF-1 polypeptide marker is determined by: (a) contacting said sample with a binding moiety which binds specifically to said RSF-1 polypeptide or fragment thereof to produce a binding moiety-RSF-1 polypeptide complex, and (b) detecting the binding moiety-RSF-1 polypeptide complex, wherein the presence of said complex is indicative of cancer, e.g., breast or ovarian cancer.

In one embodiment, the binding moiety is an antibody or a fragment thereof. In one embodiment, the antibody is a monoclonal antibody. In another embodiment, the antibody is a polyclonal antibody. In another embodiment the antibody further comprises a label. In one embodiment, the label is selected from the group consisting of a radioactive label, a fluorescent label, a chemiluminescent label, a spin label, a colored label, and an enzymatic label. In one embodiment, the method for detecting the presence of a RSF-1 polypeptide further comprises the step of measuring the concentration or amount of the polypeptide in the sample.

In one embodiment, the protein may be reacted with a binding moiety, such as an antibody, capable of specifically binding the protein being detected. Binding moieties, such as antibodies, may be designed using methods available in the art so that they interact specifically with the protein being detected. Optionally, a labeled binding moiety may be utilized. In such an embodiment, the sample is reacted with a labeled binding moiety capable of specifically binding the protein, such as a labeled antibody, to form a labeled complex of the binding moiety and the target protein being detected. Detection of the presence of the labeled complex then may provide an indication of the presence of a breast cancer in the individual being tested.

In one approach, for example, the marker protein may be detected using a binding moiety capable of specifically binding the marker protein. The binding moiety may comprise, for example, a member of a specific binding pair, such as antibody-antigen, enzyme-substrate, nucleic acid-nucleic acid, protein-nucleic acid, protein-protein, or other specific binding pair known in the art. Binding proteins may be designed which have enhanced affinity for a target protein. Optionally, the binding moiety may be linked with a detectable label, such as an enzymatic, fluorescent, radioactive, phosphorescent or colored particle label. The labeled complex may be detected, e.g., visually or with the aid of a spectrophotometer or other detector.

A RSF-1 marker may be detected using any of a wide range of immunoassay techniques available in the art. For example, the skilled artisan may employ the sandwich immunoassay format to detect breast cancer in a body fluid sample. Alternatively, the skilled artisan may use conventional immuno-histochemical procedures for detecting the presence of RSF-1 polypeptide a tissue sample using one or more labeled binding proteins.

In a sandwich immunoassay, two antibodies capable of binding the marker protein generally are used, e.g., one immobilized onto a solid support, and one free in solution and labeled with a detectable chemical compound. Examples of chemical labels that may be used for the second antibody include radioisotopes, fluorescent compounds, spin labels, colored particles such as colloidal gold and colored latex, and enzymes or other molecules that generate colored or electrochemically active products when exposed to a reactant or enzyme substrate. When a sample containing the marker protein is placed in this system, the marker protein binds to both the immobilized antibody and the labeled antibody, to form a “sandwich” immune complex on the support's surface. The complexed protein is detected by washing away non-bound sample components and excess labeled antibody, and measuring the amount of labeled antibody complexed to protein on the support's surface. Alternatively, the antibody free in solution, which can be labeled with a chemical moiety, for example, a hapten, may be detected by a third antibody labeled with a detectable moiety which binds the free antibody or, for example, the hapten coupled thereto.

Both the sandwich immunoassay and tissue immunohistochemical procedures are highly specific and very sensitive, provided that labels with good limits of detection are used. A detailed review of immunological assay design, theory and protocols can be found in numerous texts in the art, including Butt, W. R., ed. (1984) Practical Immunology, Marcel Dekker, N.Y. and Harlow et al. eds. (1988) Antibodies, A Laboratory Approach, Cold Spring Harbor Laboratory.

In general, immunoassay design considerations include preparation of antibodies (e.g., monoclonal or polyclonal antibodies) having sufficiently high binding specificity for the target protein to form a complex that can be distinguished reliably from products of nonspecific interactions. As used herein, the term “antibody” is understood to mean binding proteins, for example, antibodies or other proteins comprising an immunoglobulin variable region-like binding domain, having the appropriate binding affinities and specificities for the target protein. The higher the antibody binding specificity, the lower the target protein concentration that can be detected.

Antibodies to an isolated RSF-1 polypeptide which are useful in assays for detecting a cancer in an individual may be generated using standard immunological procedures well known and described in the art. See, for example, Practical Immunology, Butt, N. R., ed., Marcel Dekker, NY, 1984. Briefly, an isolated target protein is used to raise antibodies in a xenogeneic host, such as a mouse, goat or other suitable mammal. The marker protein is combined with a suitable adjuvant capable of enhancing antibody production in the host, and is injected into the host, for example, by intraperitoneal administration. Any adjuvant suitable for stimulating the host's immune response may be used. A commonly used adjuvant is Freund's complete adjuvant (an emulsion comprising killed and dried microbial cells). Where multiple antigen injections are desired, the subsequent injections may comprise the antigen in combination with an incomplete adjuvant (e.g., cell-free emulsion). Polyclonal antibodies may be isolated from the antibody-producing host by extracting serum containing antibodies to the protein of interest. Monoclonal antibodies may be produced by isolating host cells that produce the desired antibody, fusing these cells with myeloma cells using standard procedures known in the immunology art, and screening for hybrid cells (hybridomas) that react specifically with the target protein and have the desired binding affinity.

Antibody binding domains also may be produced biosynthetically and the amino acid sequence of the binding domain manipulated to enhance binding affinity with a preferred epitope on the target protein. Specific antibody methodologies are well understood and described in the literature. A more detailed description of their preparation can be found, for example, in Butt (1984) (supra).

In addition, genetically engineered biosynthetic antibody binding sites, also known in the art as BABS or sFv's, may be used in the practice of the instant invention. Methods for making and using BABS comprising (i) non-covalently associated or disulfide bonded synthetic VH and VL dimers, (ii) covalently linked VH-VL single chain binding sites, (iii) individual VH or VL domains, or (iv) single chain antibody binding sites are disclosed, for example, in U.S. Pat. Nos. 5,091,513; 5,132,405; 4,704,692; and 4,946,778. Furthermore, BABS having requisite specificity for the RSF-1 polypeptide can be derived by phage antibody cloning from combinatorial gene libraries (see, for example, Clackson et al. (1991) Nature 352: 624-628; or U.S. Pat. No. 5,837,500). Briefly, phage each expressing on their coat surfaces BABS having immunoglobulin variable regions encoded by variable region gene sequences derived from mice pre-immunized with RSF-1 polypeptide, or fragments thereof, are screened for binding activity against immobilized RSF-1 polypeptide. Phage which bind to the immobilized RSF-1 polypeptide are harvested and the gene encoding the BABS is sequenced. The resulting nucleic acid sequences encoding the BABS of interest then may be expressed in conventional expression systems to produce the BABS protein.

Marker proteins may also be detected using gel electrophoresis techniques available in the art. In two-dimensional gel electrophoresis, the proteins are separated first in a pH gradient gel according to their isoelectric point. The resulting gel then is placed on a second polyacrylamide gel, and the proteins separated according to molecular weight (see, for example, O'Farrell (1975) J. Biol. Chem. 250: 4007-4021; or Berkelman et al. (October 1998) 2-D Electrophoresis Using Immobilized pH Gradients: Principles and Methods, Amersham Pharmacia Biotech Pub. 80-6429-60, Rev. A).

One or more marker proteins may be detected by first isolating proteins from a sample obtained from an individual suspected of having cancer, and then separating the proteins by two-dimensional gel electrophoresis to produce a characteristic two-dimensional gel electrophoresis pattern. The pattern may then be compared with a standard gel pattern produced by separating, under the same or similar conditions, proteins isolated from normal or cancer cells. The standard gel pattern may be stored in, and retrieved from an electronic database of electrophoresis patterns. The presence of a RSF-1 polypeptide in the two-dimensional gel provides an indication that the sample being tested was taken from a person with cancer, e.g., ovarian cancer. As with the other detection assays described herein, the detection of two or more proteins, for example, in the two-dimensional gel electrophoresis pattern further enhances the accuracy of the assay. The assay thus permits the early detection and treatment of cancer.

Mass spectrometry may also be used to detect a marker protein. Preferred mass spectrometry methods include MALDI-TOF mass spectrometry and MALDI-TOF using derivatized chip surfaces (SELDI). Useful mass spectrometry methods for detecting a marker protein are described, for example, in the Examples and in U.S. Pat. Nos. 5,719,060; 6,124,137; 6,207,370; 6,225,047; 6,281,493; and 6,322,970.

These detection methods may be used in combination with each other, with other detection methods, and/or with one or more purification methods to reduce the complexity of a biological sample. Thus, for example, proteins isolated by two-dimensional gel electrophoresis could be probed with an antibody that specifically binds the marker protein, or could be assayed by mass spectrometry. Similarly, as described in the Examples, a biological sample may be subjected to biochemical fractionation prior to analysis by mass spectrometry or by other techniques such as gel electrophoresis and/or immunoassays. A marker protein may also be detected indirectly, for example, by subjecting it to enzymatic treatment, and subsequently detecting the products of that treatment.

Nucleic Acid Based Assays

In another embodiment, the RSF-1 marker is a nucleic acid encoding RSF-1 or a fragment thereof. A nucleic acid encoding RSF-1 can be detected using any method available in the art of subset of which is discussed below.

In one embodiment, the presence of a RSF-1 nucleic acid marker is detected by a nucleic acid probe which may be designed using standard methods and are used to identify DNA or mRNA encoding RSF-1. See, e.g., Maniatis et al., Molecular Cloning: A Laboratory Manual, Cold Spring Harbor Press (1989). In one embodiment, the nucleic acid probe is complementary to at least a portion of a DNA or RNA encoding a RSF-1 polypeptide.

In one embodiment, the nucleic acid probe capable of detecting RSF-1 is in a microarray containing a plurality of probes.

A detecting step according to the invention may comprise amplifying nucleic acid encoding a RSF-1 polypeptide using a polymerase chain reaction (“PCR”) or a reverse-transcriptase polymerase chain reaction. Detection of products of the PCR may be accomplished using known techniques, including hybridization with nucleic acid probes complementary to the amplified sequence.

Gene probes comprising complementary RNA or, preferably, DNA to RSF-1 nucleotide sequences or mRNA sequences encoding RSF-1 polypeptides may be produced using established recombinant techniques or oligonucleotide synthesis. The probes hybridize with complementary nucleic acid sequences presented in the test specimen, and can provide exquisite specificity. A short, well-defined probe, coding for a single unique sequence is most precise and preferred. Larger probes are generally less specific. While an oligonucleotide of any length may hybridize to an mRNA transcript, oligonucleotides typically within the range of 8-100 nucleotides, preferably within the range of 15-50 nucleotides, are envisioned to be most useful in standard hybridization assays. Choices of probe length and sequence allow one to choose the degree of specificity desired. Hybridization is carried out at from 50 to 65 degrees C. in a high salt buffer solution, formamide or other agents to set the degree of complementarity required. Furthermore, the state of the art is such that -probes can be manufactured to recognize essentially any DNA or RNA sequence. For additional particulars, see, for example, Berger et al. (1987) Guide to Molecular Techniques (Methods of Enzymology, vol. 152).

A wide variety of different labels coupled to the probes or antibodies may be employed in the assays. The labeled reagents may be provided in solution or coupled to an insoluble support, depending on the design of the assay. The various conjugates may be joined covalently or noncovalently, directly or indirectly. When bonded covalently, the particular linkage group will depend upon the nature of the two moieties to be bonded. A large number of linking groups and methods for linking are taught in the literature. Broadly, the labels may be divided into the following categories: chromogens; catalyzed reactions; chemiluminescence; radioactive labels; and colloidal-sized colored particles. The chromogens include compounds which absorb light in a distinctive range so that a color may be observed, or emit light when irradiated with light of a particular wavelength or wavelength range, e.g., fluorescers. Both enzymatic and nonenzymatic catalysts may be employed. In choosing an enzyme, there will be many considerations including the stability of the enzyme, whether it is normally present in samples of the type for which the assay is designed, the nature of the substrate, and the effect if any of conjugation on the enzyme's properties. Potentially useful enzyme labels include oxiodoreductases, transferases, hydrolases, lyases, isomerases, ligases, or synthetases. Interrelated enzyme systems may also be used. A chemiluminescent label involves a compound that becomes electronically excited by a chemical reaction and may then emit light that serves as a detectable signal or donates energy to a fluorescent acceptor. Radioactive labels include various radioisotopes found in common use such as the unstable forms of hydrogen, iodine, phosphorus or the like. Colloidal-sized colored particles involve material such as colloidal gold that, in aggregate, form a visually detectable distinctive spot corresponding to the site of a substance to be detected. Additional information on labeling technology is disclosed, for example, in U.S. Pat. No. 4,366,241.

A common method of in vitro labeling of nucleotide probes involves nick translation wherein the unlabeled DNA probe is nicked with an endonuclease to produce free 3′ hydroxyl termini within either strand of the double-stranded fragment. Simultaneously, an exonuclease removes the nucleotide residue from the 5′ phosphoryl side of the nick. The sequence of replacement nucleotides is determined by the sequence of the opposite strand of the duplex. Thus, if labeled nucleotides are supplied, DNA polymerase will fill in the nick with the labeled nucleotides. Using this well-known technique, up to 50% of the molecule can be labeled. For smaller probes, known methods involving ₃′ end labeling may be used. Furthermore, there are currently commercially available methods of labeling DNA with fluorescent molecules, catalysts, enzymes, or chemiluminescent materials. Biotin labeling kits are commercially available (Enzo Biochem Inc.) under the trademark Bio-Probe. This type of system permits the probe to be coupled to avidin which in turn is labeled with, for example, a fluorescent molecule, enzyme, antibody, etc. For further disclosure regarding probe construction and technology, see, for example, Sambrook et al. (1989) supra, or Wu et al. (1997) Methods In Gene Biotechnology, CRC Press, New York.

The oligonucleotide selected for hybridizing to the target nucleic acid, whether synthesized chemically or by recombinant DNA methodologies, may be isolated and purified using standard techniques.and then preferably labeled (e.g., with ³⁵S or ³²P) using standard labeling protocols. A sample containing the target nucleic acid then is run on an electrophoresis gel, the dispersed nucleic acids transferred to a nitrocellulose filter and the labeled oligonucleotide exposed to the filter under stringent hybridizing conditions, e.g., 50% formamide, 5. times.SSPE, 2. times. Denhardt's solution, 0.1% SDS at 42° C., as described in Sambrook et al. (1989) supra. The filter may then be washed using 2×SSPE, 0.1% SDS at 68° C., and more preferably using 0.1×SSPE, 0.1% SDS at 68° C. Other useful procedures known in the art include solution hybridization, and dot and slot RNA hybridization. Optionally, the amount of the target nucleic acid present in a sample is then quantitated by measuring the radioactivity of hybridized fragments, using standard procedures known in the art.

Nucleic acid in a sample may also be detected by, for example, a Southern blot analysis by reacting the sample with a labeled hybridization probe, wherein the probe is capable of hybridizing specifically with at least a portion of the target nucleic acid molecule. Nucleic acid in a sample may also be detected by Northern blot analysis. A nucleic acid binding protein may also be used to detect nucleic acid encoding breast cancer-associated proteins.

Kits

In one embodiment, the invention provides a kit for detecting a cell-proliferative disorder comprising an agent which binds specifically to a RSF-1 marker and instructions for use.

In one embodiment, the kit may comprise a reference sample, e.g., a negative and/or positive control. In that embodiment, the negative control would be indicative of a normal cell type and the positive control would be indicative of cancer. Such a kit may also be used for identifying potential candidate therapeutic agents for treating cancer. In one embodiment, the first binding moiety is labeled. In one embodiment, the kit further comprises a second binding moiety which binds specifically to the first binding moiety.

The above mentioned kit can be used for the detection of any cell-proliferative cancer including, without limitation, breast cancer, ovarian cervical cancer, prostate cancer, colon cancer, lung cancer, skin cancer, leukemia, lymphoma, melanoma or any other type of cancer. In one embodiment the kit is for the detection of ovarian cancer.

The kit may also be used to determine the aggressiveness of cancer, e.g., ovarian cancer.

In one embodiment, the binding moiety in the kit is an antibody or fragment thereof which specifically binds to RSF-1. Antibodies and binding fragments thereof can be lyophilized or in solution. Additionally, the preparations can contain stabilizers to increase the shelf-life of the kits, e.g., bovine serum albumin (BSA). Wherein the antibodies and antigen binding fragments thereof are lyophilized, the kit can contain further preparations of solutions to reconstitute the preparations. Acceptable solutions are well known in the art, e.g., PBS. In one embodiment, the antibody is a polyclonal antibody, a monoclonal antibody, a humanized antibody, a chimeric antibody, a recombinant antibody, or fragment thereof. In a preferred embodiment, the antibody, or fragment thereof is immunoreactive with the extracellular domain of RSF-1 or with soluble RSF-1.

Kits of the present invention can further include the components for an ELISA assay for measuring RSF-1 and fragments thereof. Samples to be tested in this application include, for example, blood, serum, plasma, urine, lymph, tissue and products thereof.

Alternatively, the kits are used in immunoassays, such as immunohistochemistry to test subject tissue biopsy sections. The kits may also be used to detect the presence of a RSF-1 marker in a biological sample obtained from a subject using immunohistocytochemistry.

The compositions of the kit of the present invention can be formulated in single or multiple units for either a single test or multiple tests.

Methods of Monitoring Therapy

In one embodiment, the invention comprises a method of monitoring the effectiveness of a treatment for a cell-proliferative disorder in a mammal, comprising quantifying the amount of a RSF-1 marker in a sample, wherein a decrease in the RSF-1 marker is indicative of the effectiveness of the treatment. The above-described method can be used to monitor the effectiveness of a cancer treatment. In a preferred embodiment, the method is used to monitor the effectiveness of ovarian cancer treatment.

In one embodiment, the concentration of a RSF-1 polypeptide or fragment thereof is compared to a standard sample obtained from healthy and/or untreated subject. Samples can be collected at discrete intervals during treatment and compared to the standard. It is contemplated that changes in the level of RSF-1 will be indicative of the efficacy of treatment.

Where the assay is used to monitor progression of a cell-proliferative disorder such as cancer or the efficacy of a treatment, the step of detecting the presence and abundance of the marker protein or its transcript in samples of interest is repeated at intervals and these values then are compared, the changes in the detected concentrations reflecting changes in the status of the tissue. For example, an increase in the level of RSF-1 may correlate with progression of the cancer. Where the assay is used to evaluate the efficacy of a therapy, the monitoring steps occur following administration of the therapeutic agent or procedure (e.g., following administration of a chemotherapeutic agent or following radiation treatment). Similarly, a decrease in the level of RSF-1 may correlate with a regression of the cancer.

Thus, cancer may be identified by the presence of RSF-1 as taught herein. Once identified, the cancer may be treated using compounds that reduce in vivo the expression and/or biological activity of the RSF-1. Furthermore, the methods provided herein can be used to monitor the progression and/or treatment of the disease.

Methods of Treatment

Because RSF-1 is present at detectably higher levels in cancer cells, e.g., ovarian cancer cells, relative to normal cells RSF-1 may be used as target molecule for cell-proliferative disorders in which RSF-1 is upregulated. Moreover, as demonstrated in Example 2, RSF-1 expression is strongly correlated with survival in cancer, e.g., ovarian cancer, and can therefore be used as a prognostic marker or to determine the course of treatment for a subject, i.e., the type, amount and timing of the cancer therapy.

In on embodiment, the invention provides methods and compositions for treating a cell-proliferative disorder. In a preferred embodiment the cell-proliferative disorder is cancer. In a more preferred embodiment, the cancer is ovarian cancer. In one embodiment, the invention further comprises administering a chemotherapeutic agent.

In another embodiment, the invention provides a method of treating a cell-proliferative disorder in a mammal, comprising administering to the mammal an effective amount of pharmaceutical composition comprising a RSF-1 antagonist.

In one embodiment, the invention provides a method of treating a cell-proliferative disorder in a mammal, comprising administering to the mammal an effective amount of a compound which binds specifically to a RSF-1 polypeptide to inactive or reduce the biological activity of RSF-1.

In one embodiment, the invention provides a method of treating cancer in a mammal, comprising administering to the mammal an effective amount of the antibody or fragment thereof which binds specifically to a RSF-1 polypeptide. In one embodiment, the invention provides a method of treating cancer in a mammal, comprising administering to the mammal an effective amount of the antibody or fragment thereof which binds specifically to a RSF-1 polypeptide. In one embodiment, the antibody or fragment thereof inactivates or reduces the biological activity of the protein.

In one embodiment, the invention provides a method of treating a cell-proliferative disorder in a mammal, comprising administering to the mammal an effective amount of a small molecule, for example, a small organic molecule which inhibits or reduces the biological activity of RSF-1.

In one embodiment, the invention provides a method of treating a cell-proliferative disorder in a mammal, comprising administering to the mammal an effective amount of a compound that modulates the expression of RSF-1 polypeptide. In one embodiment, the invention provides a method of treating cancer in a mammal, comprising administering to the mammal an effective amount of a compound that modulates the expression a RSF-1 polypeptide.

In one embodiment, the invention provides a method of modulating a cell-proliferative disorder in a subject comprising modulating the expression of a RSF-1 polypeptide in vivo. In a preferred embodiment the cell-proliferative disorder is cancer. In one embodiment, the cancer is breast cancer. In one embodiment, the modulating of the expression of a RSF-1 polypeptide comprises contacting a cell with a nucleic acid selected from the group consisting of a siRNA, an shRNA, an antisense nucleic acid or a ribozyme.

A cancer therapeutic of the invention is an oligonucleotide or peptide nucleic acid sequence complementary and capable of hybridizing under physiological conditions to part, or all, of the gene encoding the marker protein or to part, or all, of the transcript encoding the marker protein thereby to reduce or inhibit transcription and/or translation of the marker protein gene. Alternatively, the same technologies may be applied to reduce or inhibit transcription and/or translation of a RSF-1 polypeptide or a protein which interacts with a RSF-1 polypeptide.

Antisense oligonucleotides are relatively short nucleic acids that are complementary (or antisense) to the coding strand (sense strand) of the mRNA encoding a particular protein. Although antisense oligonucleotides are typically RNA based, they can also be DNA based. Additionally, antisense oligonucleotides are often modified to increase their stability.

The oligonucleotides can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide may include other appended groups such as peptides (e.g., for targeting host cell receptors), or agents facilitating transport across the cell membrane (see, e.g., Letsinger et al., 1989, Proc. Natl. Acad. Sci. U.S.A. 86:6553-6556; Lemaitre et al., 1987, Proc. Natl. Acad. Sci. 84:648-652; PCT Publication No. W088/09810, published Dec. 15, 1988) or the blood-brain barrier (see, e.g., PCT Publication No. W089/10134, published Apr.25, 1988), hybridization-triggered cleavage agents (See, e.g., Krol et al., 1988, BioTechniques 6:958-976) or intercalating agents. (See, e.g., Zon, 1988, Pharm. Res. 5:539-549). To this end, the oligonucleotide may be conjugated to another molecule.

The antisense oligonucleotide may comprise at least one modified base moiety which is selected from the group including but not limited to 5-fluorouracil, 5-bromouracil, 5-chlorouracil, 5-iodouracil, hypoxanthine, xanthine, 4-acetylcytosine, 5-(carboxyhydroxytriethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methyl ester, uracil-5-oxyacetic acid (v), 5-methyl-2-thiouracil, 3-(3-amino-3-N-2-carboxypropyl) uracil, (acp3)w, and 2,6-diaminopurine.

The antisense oligonucleotide may also comprise at least one modified sugar moiety selected from the group including but not limited to arabinose, 2-fluoroarabinose, xylulose, and hexose.

The antisense oligonucleotide can also contain a neutral peptide-like backbone. Such molecules are termed peptide nucleic acid (PNA)-oligomers and are described, e.g., in Perry-O'Keefe et al. (1996) Proc. Natl. Acad. Sci. U.S.A. 93:14670 and in Eglom et al. (1993) Nature 365:566. One advantage of PNA oligomers is their capability to bind to complementary DNA essentially independently from the ionic strength of the medium due to the neutral backbone of the DNA. In yet another embodiment, the antisense oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In yet a further embodiment, the antisense oligonucleotide is an -anomeric oligonucleotide. An -anomeric oligonucleotide forms specific double-stranded hybrids with complementary RNA in which, contrary to the usual units, the strands run parallel to each other (Gautier et al., 1987, Nucl. Acids Res. 15:6625-6641). The oligonucleotide is a 2′-0-methylribonucleotide (Inoue et al., 1987, Nucl. Acids Res. 15:6131-6148), or a chimeric RNA-DNA analogue (Inoue et al., 1987, FEBS Lett. 215:327-330).

Oligonucleotides of the invention may be synthesized by standard methods known in the art, e.g., by use of an automated DNA synthesizer (such as are commercially available from Biosearch, Applied Biosystems, etc.). As examples, phosphorothioate oligonucleotides may be synthesized by the method of Stein et al. (1988, Nucl. Acids Res. 16:3209), methylphosphonate oligonucleotides can be prepared by use of controlled pore glass polymer supports (Sarin et al., 1988, Proc. Natl. Acad. Sci. U.S.A. 85:7448-7451), etc.

The selection of an appropriate oligonucleotide can be readily performed by one of skill in the art. Given the nucleic acid sequence encoding a particular protein, one of skill in the art can design antisense oligonucleotides that bind to that protein, and test these oligonucleotides in an in vitro or in vivo system to confirm that they bind to and mediate the degradation of the mRNA encoding the particular protein. To design an antisense oligonucleotide that specifically binds to and mediates the degradation of a particular protein, it is important that the sequence recognized by the oligonucleotide is unique or substantially unique to that particular protein. For example, sequences that are frequently repeated across protein may not be an ideal choice for the design of an oligonucleotide that specifically recognizes and degrades a particular message. One of skill in the art can design an oligonucleotide, and compare the sequence of that oligonucleotide to nucleic acid sequences that are deposited in publicly available databases to confirm that the sequence is specific or substantially specific for a particular protein.

In another example, it may be desirable to design an antisense oligonucleotide that binds to and mediates the degradation of more than one message. In one example, the messages may encode related protein such as isoforms or functionally redundant protein. In such a case, one of skill in the art can align the nucleic acid sequences that encode these related proteins, and design an oligonucleotide that recognizes both messages.

A number of methods have been developed for delivering antisense DNA or RNA to cells; e.g., antisense molecules can be injected directly into the tissue site, or modified antisense molecules, designed to target the desired cells (e.g., antisense linked to peptides or antibodies that specifically bind receptors or antigens expressed on the target cell surface) can be administered systematically.

However, it may be difficult to achieve intracellular concentrations of the antisense sufficient to suppress translation on endogenous mRNAs in certain instances. Therefore another approach utilizes a recombinant DNA construct in which the antisense oligonucleotide is placed under the control of a strong pol III or pol II promoter. For example, a vector can be introduced in vivo such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequence encoding the antisense RNA can be by any promoter known in the art to act in mammalian, preferably human cells. Such promoters can be inducible or constitutive. Such promoters include but are not limited to: the SV40 early promoter region (Bemoist and Chambon, 1981, Nature 290:304-310), the promoter contained in the 3′ long terminal repeat of Rous sarcoma virus (Yamamoto et al., 1980, Cell 22:787-797), the herpes thymidine kinase promoter (Wagner et al., 1981, Proc. Natl. Acad. Sci. U.S.A. 78:1441-1445), the regulatory sequences of the metallothionein gene (Brinster et al, 1982, Nature 296:39-42), etc. Any type of plasmid, cosmid, YAC or viral vector can be used to prepare the recombinant DNA construct that can be introduced directly into the tissue site. Alternatively, viral vectors can be used which selectively infect the desired tissue, in which case administration may be accomplished by another route (e.g., systematically).

RNAi constructs comprise double stranded RNA that can specifically block expression of a target gene. “RNA interference” or “RNAi” is a term initially applied to a phenomenon observed in plants and worms where double-stranded RNA (dsRNA) blocks gene expression in a specific and post-transcriptional manner. Without being bound by theory, RNAi appears to involve mRNA degradation, however the biochemical mechanisms are currently an active area of research. Despite some mystery regarding the mechanism of action, RNAi provides a useful method of inhibiting gene expression in vitro or in vivo.

As used herein, the term “dsRNA” refers to siRNA molecules, or other RNA molecules including a double stranded feature and able to be processed to siRNA in cells, such as hairpin RNA moieties.

The term “loss-of-function,” as it refers to genes inhibited by the subject RNAi method, refers to a diminishment in the level of expression of a gene when compared to the level in the absence of RNAi constructs.

As used herein, the phrase “mediates RNAi” refers to (indicates) the ability to distinguish which RNAs are to be degraded by the RNAi process, e.g., degradation occurs in a sequence-specific manner rather than by a sequence-independent dsRNA response, e.g., a PKR response.

As used herein, the term “RNAi construct” is a generic term used throughout the specification to include small interfering RNAs (siRNAs), hairpin RNAs, and other RNA species which can be cleaved in vivo to form siRNAs. RNAi constructs herein also include expression vectors (also referred to as RNAi expression vectors) capable of giving rise to transcripts which form dsRNAs or hairpin RNAs in cells, and/or transcripts which can produce siRNAs in vivo.

“RNAi expression vector” (also referred to herein as a “dsRNA-encoding plasmid”) refers to replicable nucleic acid constructs used to express (transcribe) RNA which produces siRNA moieties in the cell in which the construct is expressed. Such vectors include a transcriptional unit comprising an assembly of (1) genetic element(s) having a regulatory role in gene expression, for example, promoters, operators, or enhancers, operatively linked to (2) a “coding” sequence which is transcribed to produce a double-stranded RNA (two RNA moieties that anneal in the cell to form an siRNA, or a single hairpin RNA which can be processed to an siRNA), and (3) appropriate transcription initiation and termination sequences. The choice of promoter and other regulatory elements generally varies according to the intended host cell. In general, expression vectors of utility in recombinant DNA techniques are often in the form of “plasmids” which refer to circular double stranded DNA loops which, in their vector form are not bound to the chromosome. In the present specification, “plasmid” and “vector” are used interchangeably as the plasmid is the most commonly used form of vector. However, the invention is intended to include such other forms of expression vectors which serve equivalent functions and which become known in the art subsequently hereto.

The RNAi constructs contain a nucleotide sequence that hybridizes under physiologic conditions of the cell to the nucleotide sequence of at least a portion of the mRNA transcript for the gene to be inhibited (i.e., the “target” gene). The double-stranded RNA need only be sufficiently similar to natural RNA that it has the ability to mediate RNAi. Thus, the invention has the advantage of being able to tolerate sequence variations that might be expected due to genetic mutation, strain polymorphism or evolutionary divergence. The number of tolerated nucleotide mismatches between the target sequence and the RNAi construct sequence is no more than 1 in 5 basepairs, or 1 in 10 basepairs, or 1 in 20 basepairs, or 1 in 50 basepairs. Mismatches in the center of the siRNA duplex are most critical and may essentially abolish cleavage of the target RNA. In contrast, nucleotides at the 3′ end of the siRNA strand that is complementary to the target RNA do not significantly contribute to specificity of the target recognition.

Sequence identity may be optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group). Greater than 90% sequence identity, or even 100% sequence identity, between the inhibitory RNA and the portion of the target gene is preferred. Alternatively, the duplex region of the RNA may be defined functionally as a nucleotide sequence that is capable of hybridizing with a portion of the target gene transcript (e.g., 400 mM NaCl, 40 mM PIPES pH 6.4, 1 mM EDTA, 50° C. or 70° C. hybridization for 12-16 hours; followed by washing).

Production of RNAi constructs can be carried out by chemical synthetic methods or by recombinant nucleic acid techniques. Endogenous RNA polymerase of the treated cell may mediate transcription in vivo, or cloned RNA polymerase can be used for transcription in vitro. The RNAi constructs may include modifications to either the phosphate-sugar backbone or the nucleoside, e.g., to reduce susceptibility to cellular nucleases, improve bioavailability, improve formulation characteristics, and/or change other pharmacokinetic properties. For example, the phosphodiester linkages of natural RNA may be modified to include at least one of a nitrogen or sulfur heteroatom. Modifications in RNA structure may be tailored to allow specific genetic inhibition while avoiding a general response to dsRNA. Likewise, bases may be modified to block the activity of adenosine deaminase. The RNAi construct may be produced enzymatically or by partial/total organic synthesis, any modified ribonucleotide can be introduced by in vitro enzymatic or organic synthesis.

Methods of chemically modifying RNA molecules can be adapted for modifying RNAi constructs (see, for example, Heidenreich et al. (1997) Nucleic Acids Res, 25:776-780; Wilson et al. (1994) J Mol Recog 7:89-98; Chen et al. (1995) Nucleic Acids Res 23:2661-2668; Hirschbein et al. (1997) Antisense Nucleic Acid Drug Dev 7:55-61). Merely to illustrate, the backbone of an RNAi construct can be modified with phosphorothioates, phosphoramidate, phosphodithioates, chimeric methylphosphonate-phosphodiesters, peptide nucleic acids, 5-propynyl-pyrimidine containing oligomers or sugar modifications (e.g., 2′-substituted ribonucleosides, a-configuration).

The double-stranded structure may be formed by a single self-complementary RNA strand or two complementary RNA strands. RNA duplex formation may be initiated either inside or outside the cell. The RNA may be introduced in an amount which allows delivery of at least one copy per cell. Higher doses (e.g., at least 5, 10, 100, 500 or 1000 copies per cell) of double-stranded material may yield more effective inhibition, while lower doses may also be useful for specific applications. Inhibition is sequence-specific in that nucleotide sequences corresponding to the duplex region of the RNA are targeted for genetic inhibition.

In certain embodiments, the subject RNAi constructs are “small interfering RNAs” or “siRNAs.” These nucleic acids are around 19-30 nucleotides in length, and even more preferably 21-23 nucleotides in length, e.g., corresponding in length to the fragments generated by nuclease “dicing” of longer double-stranded RNAs. The siRNAs are understood to recruit nuclease complexes and guide the complexes to the target mRNA by pairing to the specific sequences. As a result, the target mRNA is degraded by the nucleases in the protein complex. In a particular embodiment, the 21-23 nucleotides siRNA molecules comprise a 3′ hydroxyl group.

The siRNA molecules of the present invention can be obtained using a number of techniques known to those of skill in the art. For example, the siRNA can be chemically synthesized or recombinantly produced using methods known in the art. For example, short sense and antisense RNA oligomers can be synthesized and annealed to form double-stranded RNA structures with 2-nucleotide overhangs at each end (Caplen, et al. (2001) Proc Natl Acad Sci USA, 98:9742-9747; Elbashir, et al. (2001) EMBO J, 20:6877-88). These double-stranded siRNA structures can then be directly introduced to cells, either by passive uptake or a delivery system of choice, such as described below.

In certain embodiments, the siRNA constructs can be generated by processing of longer double-stranded RNAs, for example, in the presence of the enzyme dicer. In one embodiment, the Drosophila in vitro system is used. In this embodiment, dsRNA is combined with a soluble extract derived from Drosophila embryo, thereby producing a combination. The combination is maintained under conditions in which the dsRNA is processed to RNA molecules of about 21 to about 23 nucleotides.

The siRNA molecules can be purified using a number of techniques known to those of skill in the art. For example, gel electrophoresis can be used to purify siRNAs. Alternatively, non-denaturing methods, such as non-denaturing column chromatography, can be used to purify the siRNA. In addition, chromatography (e.g., size exclusion chromatography), glycerol gradient centrifugation, affinity purification with antibody can be used to purify siRNAs.

In certain preferred embodiments, at least one strand of the siRNA molecules has a 3′ overhang from about 1 to about 6 nucleotides in length, though may be from 2 to 4 nucleotides in length. More preferably, the 3′ overhangs are 1-3 nucleotides in length. In certain embodiments, one strand having a 3′ overhang and the other strand being blunt-ended or also having an overhang. The length of the overhangs may be the same or different for each strand. In order to further enhance the stability of the siRNA, the 3′ overhangs can be stabilized against degradation. In one embodiment, the RNA is stabilized by including purine nucleotides, such as adenosine or guanosine nucleotides. Alternatively, substitution of pyrimidine nucleotides by modified analogues, e.g., substitution of uridine nucleotide 3′ overhangs by 2′-deoxythyinidine is tolerated and does not affect the efficiency of RNAi. The absence of a 2′ hydroxyl significantly enhances the nuclease resistance of the overhang in tissue culture medium and may be beneficial in vivo.

In other embodiments, the RNAi construct is in the form of a long double-stranded RNA. In certain embodiments, the RNAi construct is at least 25, 50, 100, 200, 300 or 400 bases. In certain embodiments, the RNAi construct is 400-800 bases in length. The double-stranded RNAs are digested intracellularly, e.g., to produce siRNA sequences in the cell. However, use of long double-stranded RNAs in vivo is not always practical, presumably because of deleterious effects which may be caused by the sequence-independent dsRNA response. In such embodiments, the use of local delivery systems and/or agents which reduce the effects of interferon or PKR are preferred.

In certain embodiments, the RNAi construct is in the form of a hairpin structure (named as hairpin RNA). The hairpin RNAs can be synthesized exogenously or can be formed by transcribing from RNA polymerase III promoters in vivo. Examples of making and using such hairpin RNAs for gene silencing in mammalian cells are described in, for example, Paddison et al., Genes Dev, 2002, 16:948-58; McCaffrey et al., Nature, 2002, 418:38-9; McManus et al., RNA, 2002, 8:842-50; Yu et al., Proc Natl Acad Sci USA, 2002, 99:6047-52). Preferably, such hairpin RNAs are engineered in cells or in an animal to ensure continuous and stable suppression of a desired gene. It is known in the art that siRNAs can be produced by processing a hairpin RNA in the cell.

In yet other embodiments, a plasmid is used to deliver the double-stranded RNA, e.g., as a transcriptional product. In such embodiments, the plasmid is designed to include a “coding sequence” for each of the sense and antisense strands of the RNAi construct. The coding sequences can be the same sequence, e.g., flanked by inverted promoters, or can be two separate sequences each under transcriptional control of separate promoters. After the coding sequence is transcribed, the complementary RNA transcripts base-pair to form the double-stranded RNA.

PCT application WO01/77350 describes an exemplary vector for bi-directional transcription of a transgene to yield both sense and antisense RNA transcripts of the same transgene in a eukaryotic cell. Accordingly, in certain embodiments, the present invention provides a recombinant vector having the following unique characteristics: it comprises a viral replicon having two overlapping transcription units arranged in an opposing orientation and flanking a transgene for an RNAi construct of interest, wherein the two overlapping transcription units yield both sense and antisense RNA transcripts from the same transgene fragment in a host cell.

RNAi constructs can comprise either long stretches of double stranded RNA identical or substantially identical to the target nucleic acid sequence or short stretches of double stranded RNA identical to substantially identical to only a region of the target nucleic acid sequence. Exemplary methods of making and delivering either long or short RNAi constructs can be found, for example, in WO01/68836 and WOO01/75164.

Ribozyme molecules designed to catalytically cleave an mRNA transcript can also be used to prevent translation of mRNA (See, e.g., PCT International Publication WO90/11364, published Oct. 4, 1990; Sarver et al., 1990, Science 247:1222-1225 and U.S. Pat. No. 5,093,246). While ribozymes that cleave mRNA at site-specific recognition sequences can be used to destroy particular mRNAs, the use of hammerhead ribozymes is preferred. Hammerhead ribozymes cleave mRNAs at locations dictated by flanking regions that form complementary base pairs with the target mRNA. The sole requirement is that the target mRNA has the following sequence of two bases: 5′-UG-3′. The construction and production of hammerhead ribozymes is well known in the art and is described more fully in Haseloff and Gerlach, 1988, Nature, 334:585-591.

The ribozymes of the present invention also include RNA endoribonucleases (hereinafter “Cech-type ribozymes”) such as the one which occurs naturally in Tetrahymena thermophila (known as the IVS, or L-19 IVS RNA) and which has been extensively described by Thomas Cech and collaborators (Zaug, et al., 1984, Science, 224:574-578; Zaug and Cech, 1986, Science, 231:470475; Zaug, et al., 1986, Nature, 324:429-433; published International patent application No. WO88/04300 by University Patents Inc.; Been and Cech, 1986, Cell, 47:207-216). The Cech-type ribozymes have an eight base pair active site that hybridizes to a target RNA sequence whereafter cleavage of the target RNA takes place. The invention encompasses those Cech-type ribozymes that target eight base-pair active site sequences.

As in the antisense approach, the ribozymes can be composed of modified oligonucleotides (e.g., for improved stability, targeting, etc.) and can be delivered to cells in vitro or in vivo. A preferred method of delivery involves using a DNA construct “encoding” the ribozyme under the control of a strong constitutive pol III or pol II promoter, so that transfected cells will produce sufficient quantities of the ribozyme to destroy targeted messages and inhibit translation. Because ribozymes unlike antisense molecules, are catalytic, a lower intracellular concentration is required for efficiency.

In addition to administration with conventional carriers, the anti-sense oligonucleotides or peptide nucleic acid sequences may be administered by a variety of specialized oligonucleotide delivery techniques. For example, oligonucleotides may be encapsulated in liposomes, as described in Mannino et al. (1988) BioTechnology 6: 682, and Felgner et al. (1989) Bethesda Res. Lab. Focus 11:21. Lipids useful in producing liposomal formulations include, without limitation, monoglycerides, diglycerides, sulfatides, lysolecithin, phospholipids, saponin, bile acids, and the like. Preparation of such liposomal formulations is within the level of skill in the art (see, for example, in U.S. Pat. Nos. 4,235,871; 4,501,728; 4,837,028; and 4,737,323). The pharmaceutical composition of the invention may further include compounds such as cyclodextrins and the like which enhance delivery of oligonucleotides into cells. When the composition is not administered systemically but, rather, is injected at the site of the target cells, cationic detergents (e.g. Lipofectin) may be added to enhance uptake. In addition, reconstituted virus envelopes have been successfully used to deliver RNA and DNA to cells (see, for example, Arad et al. (1986) Biochem. Biophy. Acta 859: 88-94).

For therapeutic use in vivo, the anti-sense oligonucleotides and/or peptide nucleic acid sequences are administered to the individual in a therapeutically effective amount, for example, an amount sufficient to reduce or inhibit target protein expression in malignant cells. The actual dosage administered may take into account whether the nature of the treatment is prophylactic or therapeutic in nature, the age, weight, health of the subject, the route of administration, the size and nature of the malignancy, as well as other factors. The daily dosage may range from about 0.01 to 1,000 mg per day. Greater or lesser amounts of oligonucleotide or peptide nucleic acid sequences may be administered, as required. As will be appreciated by those skilled in the medical art, particularly the chemotherapeutic art, appropriate dose ranges for in vivo administration would be routine experimentation for a clinician. As a preliminary guideline, effective concentrations for in vitro inhibition of the target molecule may be determined first.

The skilled artisan can, using methodologies well known in the art, screen small molecule libraries (either peptide or non-peptide based libraries) to identify candidate molecules that reduce or inhibit the biological function of the RSF-1. The small molecules preferably accomplish this function by reducing the in vivo expression of the target molecule, or by interacting with the target molecule thereby to inhibit either the biological activity of the target molecule or an interaction between the target molecule and its in vivo binding partner.

It is contemplated that, once the candidate small molecules have been elucidated, the skilled artisan may enhance the efficacy of the small molecule using rational drug design methodologies well known in the art. Alternatively, the skilled artisan may use a variety of computer programs which assist the skilled artisan to develop quantitative structure activity relationships (QSAR) which further to assist the design of additional candidate molecules de novo. Once identified, the small molecules may be produced in commercial quantities and subjected to the appropriate safety and efficacy studies.

It is contemplated that the screening assays may be automated thereby facilitating the screening of a large number of small molecules at the same time. Such automation procedures are within the level of skill in the art of drug screening and, therefore, are not discussed herein. Candidate peptide-based small molecules may be produced by expression of an appropriate nucleic acid sequence in a host cell or using synthetic organic chemistries. Similarly, non-peptidyl-based small molecules may be produced using conventional synthetic organic chemistries well known in the art.

As described above, for in vivo use, the identified small molecules may be combined with a suitable pharmaceutically acceptable carrier, such as physiological saline or other useful carriers well characterized in the medical art. The pharmaceutical compositions may be provided directly to malignant cells, for example, by direct injection, or may be provided systemically, provided the binding protein is associated with means for targeting the protein to target cells. Finally, suitable dose ranges and cell toxicity levels may be assessed using standard dose range experiments. As described above, actual dosages administered may vary depending, for example, on the nature of the malignancy, the age, weight and health of the individual, as well as other factors.

One embodiment of the present invention are methods of treating a cell-proliferative disorder, e.g., cancer such as ovarian cancer, with pharmaceutical compositions of antibodies, antigen binding fragments, peptides, nucleic acids, small molecules and other compounds as described above. In a preferred embodiment, the subject receiving treatment is a human subject. Pharmaceutical compositions of the invention can be administered to a subject in need there of by, for example, injection.

Pharmaceutical compositions of the present invention are administered in a therapeutically effective amount which are effective for producing some desired therapeutic effect by inducing tumor-specific killing of tumor cells in a subject and thereby blocking the biological consequences of that pathway in the treated cells eliminating the tumor cell or preventing it from proliferating, at a reasonable benefit/risk ratio applicable to any medical treatment.

One embodiment of the present invention contemplates the use of any of the pharmaceutical compositions of the present invention to make a medicament for treating cancer. Medicaments can be formulated based on the physical characteristics of the subject/subject needing treatment, and can be formulated in single or multiple formulations based on the stage of the cancerous tissue. Medicaments of the present invention can be packaged in a suitable pharmaceutical package with appropriate labels for the distribution to hospitals and clinics wherein the label is for the indication of treating a specific cancer in a subject. Medicaments can be packaged as a single or multiple units. Instructions for the dosage and administration of the pharmaceutical compositions of the present invention can be included with the pharmaceutical packages.

In exemplary embodiments, the pharmaceutical compositions of the present invention can be administered to a subject by any convenient route, including, for example, subcutaneous, intradermal, intravenous, intra-arterial, intraperitoneal, or intramuscular injection.

In a preferred embodiment, the antibodies, antigen binding fragments, or peptides are labeled with a radiolabel or a toxin that kills the target cell upon binding of the antibodies, antigen binding fragments, or peptides to RSF-1.

In one embodiment of the present methods, the toxin is any one of ricin, ricin A chain (ricin toxin), Pseudomonas exotoxin (PE), diphtheria toxin (DT), Clostridium perfringens phospholipase C (PLC), bovine pancreatic ribonuclease (PBR), pokeweed antiviral protein (PAP), abrin, abrin A chain (abrin toxin), cobra venum factor (CVF), gelonin (GEL), saporin (SAP) modeccin, viscumin or volkensin.

Antibodies, antigen binding fragments, peptides nucleic acid molecules small molecules, and peptidomimetics of the present invention can also be used in combination therapy with chemotherapeutic agents such as the chemotherapeutic agents discussed above.

The pharmaceutical compositions can be administered separately or concomitantly. In one aspect of the present invention, the pharmaceutical compositions are administered in a single formulation. In one aspect of the present invention, the pharmaceutical compositions are administered as separate formulations.

Screening Assays

The invention also comprises methods to screen for compounds which can be used to treat a cell-proliferative disorder such as cancer.

In one embodiment, the method comprises (a) identifying a RSF-1 modulator, e.g., an antagonist, and (b) determining whether said RSF-1 antagonist is effective against a cell-proliferative disorder. Said methods can be carried out using methods which are well known in the art. For example, determining whether a RSF-1 modulator is effective against a cell-proliferative disorder can be carried out using any in vitro or in vivo models of a cell-proliferative disorder.

The invention also comprises a method to screen for RSF-1 modulators, comprising: (a) contacting a RSF-1 polypeptide with a test compound under conditions suitable for detecting the binding of the RSF-1 polypeptide to the test compound, (b) determining whether the test compound binds the RSF-1 polypeptide, and (c) further determining whether the test compound prevents, inhibits or reduces the binding of RSF-1, wherein a test compound that binds the RSF-1 polypeptide and prevents, inhibits or reduces the binding of RSF-1 is a RSF-1 antagonist. In one embodiment the method further comprises determining whether the test compound binds the extracellular domain of said RSF-1 polypeptide.

### EXAMPLES

It should be appreciated that the invention should not be construed to be limited to the examples that are now described; rather, the invention should be construed to include any and all applications provided herein and all equivalent variations within the skill of the ordinary artisan.

### Example 1

The following example demonstrates that Rsf-1 is overexpressed in ovarian cancer sample. Moreover, the examples demonstrates that subjects who overexpressed Rsf-1 expression had a significantly shorter overall survival than those without. Over-expression of Rsf-1 gene stimulated cell proliferation and transform non-neoplastic cells by conferring serum-independent and anchorage-independent growth. Furthermore, Rsf-1 gene knockdown inhibited cell growth in OVCAR3 cells, which harbor Rsf-1 amplification.

Materials and Methods

Tissue Samples

Tissue samples were obtained from the department of pathology at the Johns Hopkins Hospital between 1990 and 2004. Effusion (peritoneal and pleural) samples were obtained from the Norwegian Radium Hospital in Norway. All ovarian carcinomas were of serous type from sporadic cases. Acquisition of tissue specimens and clinical information was approved by an institutional review board (Johns Hopkins Univeristy) or by the Regional Ethics Committee (Norway).

Digital Karyotyping

Carcinoma cells were affmity purified using magnetic beads conjugated with the Epi-CAM antibody (Dynal, Oslo, Norway). The purity of tumor cells was confirmed by immunostaining with an anti-cytokeratin antibody, CAM 5.2 (Becton Dickinson, San Jose, Calif.) and samples with greater than 95% epithelial cells were used in this study. Digital karyotyping library construction and data analysis were performed following the previous protocol (6, 7). Approximate 120,000 genomic tags were obtained for each digital karyotyping library in this study. After removing the nucleotide repeats in human genome, the average of filtered tags was 66,000 for each library. We set up a window size of 300 for the analysis in this study. Based on Monte Carlo simulation, the parameters employed in this study can reliably detect >0.5 Mb amplicon with >5 fold amplification with >99% sensitivity and 100% positive predictive value.

Fluorescence In Situ Hybridization (FISH) and Immunohistochemistry

Formalin-fixed, paraffin-embedded tissues were arranged onto tissue microarrays to facilitate FISH analysis. Three representative cores (1.5 mm diameter) from each tumor were placed on the tissue microarrays. BAC clones containing the genomic sequences of the 11q13.5 amplicon at 77.05-77.23 Mb (RP11-1107J12) and EMSY at 75.88-76.09 Mb (CTD-2501F13) were purchased from Bacpac Resources (Children's Hospital Oakland, Calif.) and Invitrogen (Carlsbad, Calif.). RP11-846G12 BAC clone, located at 11q11 (55.88-56.05 Mb), was used as the control probe. The method for FISH has been detailed in a previous report (7). Two individuals who were not aware of the tumor grade and clinical information evaluated FISH signals. Approximately 100 tumor cells were examined for each specimen. Amplification of the Rsf-1 and EMSY genes was defined as a ratio of the gene probe signal to the control probe signal exceeding 2.

A mouse monoclonal anti-Rsf-1 antibody (gift from Dr. Danny Reinberg) was used in the immunohistochemistry study. Immunohistochemistry was performed by standard protocol using an EnVision+System peroxidase kit (DAKO, Carpinteria, Calif.).

Quantitative Real-Time PCR

Real-time PCR for genomic DNA copy numbers and gene expression levels was performed using methods previously described (11). PCR reactions were performed using an iCycler (Bio-Rad Lab, Hercules, Calif.). For quantitative PCR performed with genomic DNA, we used a cutoff ratio of 2.2 to define genomic amplification. This cutoff value was determined as the mean+2 standard deviations based on quantitative PCR analyses of normal diploid cells using all the primer sets. This cutoff value gave a confidence level of 97.5%.

Cell Proliferation Assay

Cells were seeded in 96-well plates at a density of 4,000 cells per well. Cell number was determined indirectly by the fluorescence intensity of SYBR®Green I nucleic acid gel stain (Molecular Probes, Eugene, Oreg. USA) using a microplate reader (Fluostar from BMG, Durham, N.C.). Data was expressed as the mean±1 standard deviation from 5 replicates in each experimental group. Anchorage-independent growth assay was performed as previously described (12). Data were expressed as the mean±1 standard deviation from triplicates.

siRNA-Mediated Knockdown of Rsf-1 Expression

Three siRNAs that targeted Rsf-1 were designed and their sense sequences were: GGAAAGACAUCUCUACUAUUU (SEQ ID NO:3), UAAAUGAUCUGGACAGUGAUU (SEQ ID NO:4) and GGACUUACCUUCAACCAAUUU (SEQ ID NO:5). Control siRNA (off-target control, cat# D-001210-02-05) was purchased from Dharmacon (Lafayette, Colo.). Cells were seeded in 96 wells and transfected with siRNAs using oligofectamine (Invitrogen, Carlsbad, Calif.). BrdU uptake and staining were performed using a cell proliferation kit (Amersham, Buckinghamshire, England). Apoptotic cells were detected using an annexin V staining kit (BioVision, Mountain View, Calif.). The percentage of BrdU-positive and annexin V-positive cells was determined by counting approximately 300 cells from each well in 96-well plates. The data was expressed as mean+1 standard deviation from triplicates.

Statistical Method for Clinical Correlation

Overall survival was calculated from the date of primary surgery for ovarian tumors to the date of death or last follow-up. Patients with Rsf-1 amplification and without amplification had similar age distributions and received optimal tumor debulking surgery followed by carboplatin and taxol-based chemotherapy. The data was plotted as Kaplan-Meier curves and the statistical significance was determined by the Log-rank test. Data were censored when patients were lost to follow-up. In a Cox proportional hazard model, the p-values were assessed using a likelihood ratio test as implemented by the “survival” package in the statistical programming language R. Student t-test was used to examine the statistical significance in difference of growth assay data.

Generation of Rsf-1 Expressing RK3E Cells

To study the functional roles of Rsf-1 overexpression, we generated stable clones of RK3E that overexpressed Rsf-1. The full-length of Rsf-1 coding region was cloned into a mammalian expression vector, pCDNA6. One million of RK3E cells were transfected with an Rsf-1 expression vector using electroporation (Amaxa, Germany). The transfectants were minimally diluted and cultured in DMEM containing 10% bovine serum and 300 mg/ml of G418 (Sigma). Stable Rsf-1 clones were selected for growth assay.

Effects of shRNA on Tumor Xenograft in Nude Mice

ShRNA sequence was subcloned into pRNAT-U6.3/hygro vector (GenScript, Piscataway, N.J.) at BamH1 and HindIII sites. The targeting sequence for Rsf-1 shRNA was: ggatcccgtctttgtctcgaccaattggcttgatatccggccaattggtcgagacaaagattttttccaaaagctt (SEQ ID NO:6). The control sequence for scramble shRNA was: ggatcccgatatcaattccttctgtccattgatatccgtggacagaaggaattgatatctttttccaaaagctt (SEQ ID NO:7). Five million of OVCAR3 cells were transfected with Rsf-1 and scramble shRNA and were selected with hygromycin (200 μg/ml) for 5 days. Positive clones (pool) were propagated and then 3 million cells were i.p. injected into nu/nu mice (6-8 weeks old), which were sacrificed after 6 weeks. Five mice were used for each group. Necropsy was performed in all mice to examine for intraperitoneal tumors which were excised and weighted.

Results

Digital Karyotyping of Ovarian Carcinomas

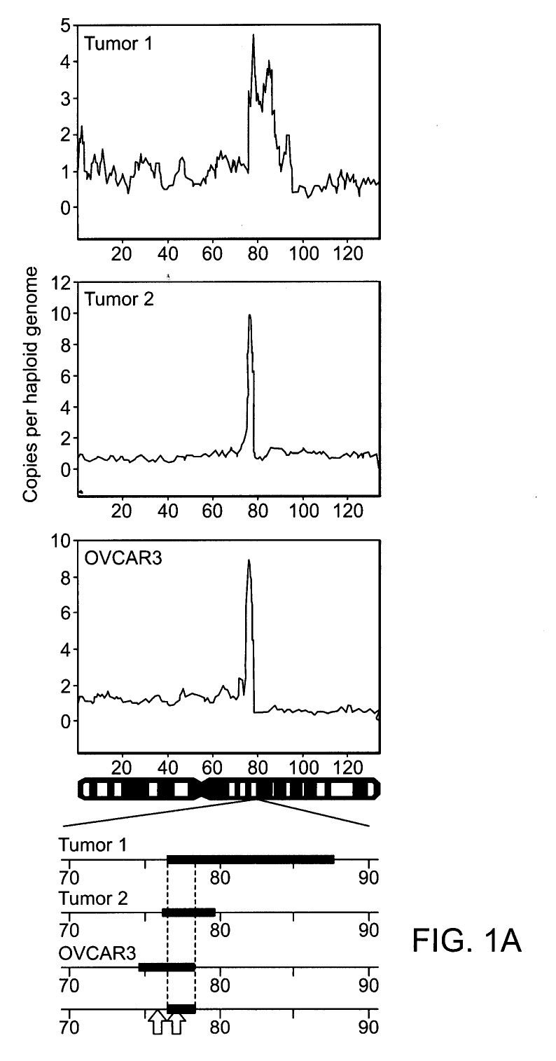

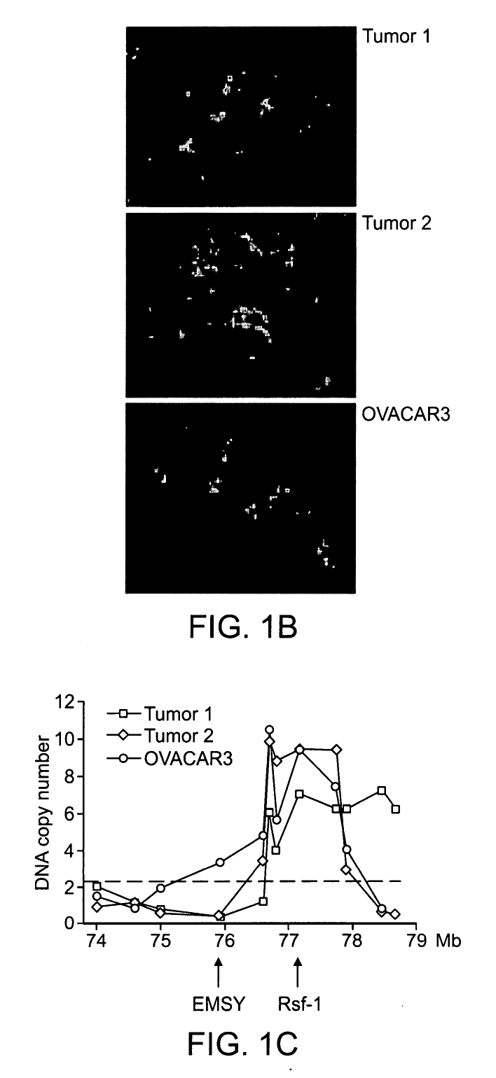

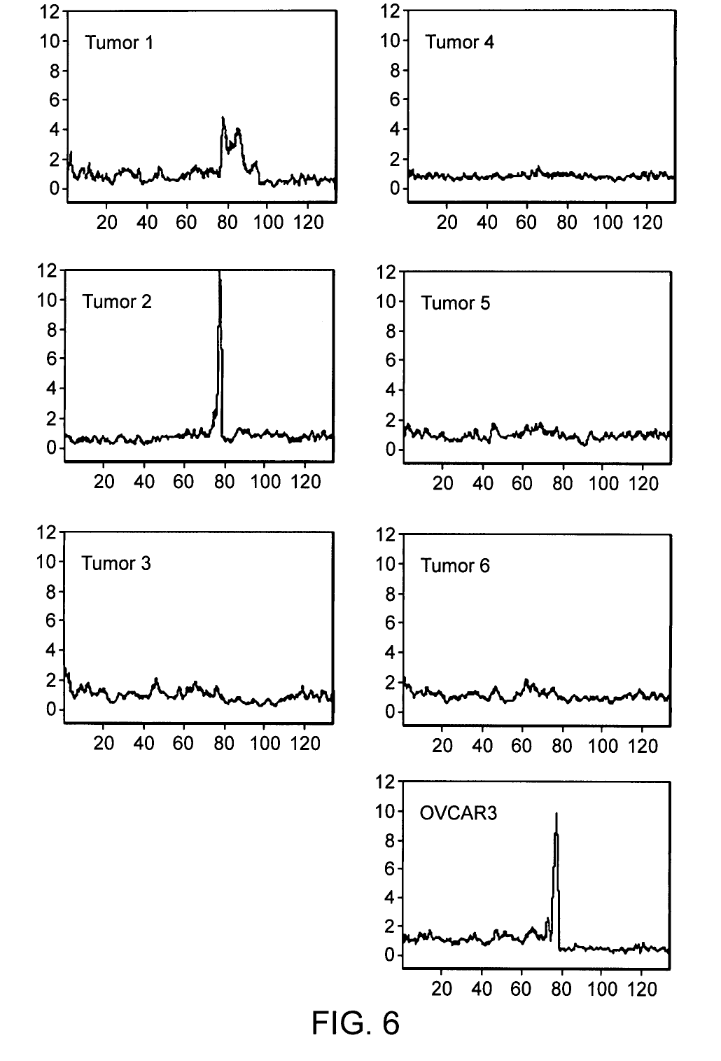

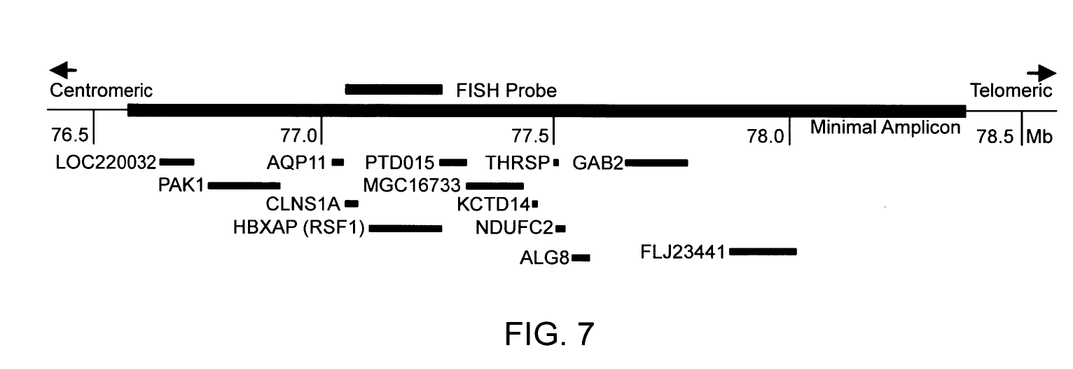

Digital karyotyping was used to evaluate the genomic alterations in 7 ovarian cancer samples including 6 high-grade ovarian serous carcinomas and one ovarian cancer cell line, OVCAR3. Analysis of the genomic tag densities along chromosomes revealed a discrete amplification at chromosome 11q13.5 in three libraries including two high-grade ovarian carcinomas and the OVCAR3 cell line. No evidence of other amplification cores was detected in chromosome 11 in any of the ovarian cancer libraries (FIG. 6). Alignment of these three amplicons delineated an overlapping region of amplification, spanning from 76.6 to 78.4 Mb on the chromosome 11q (FIG. 1a). Examination of the RefSeq database in the human genome assembly (July 2003 freeze, UCSC) revealed that 13 genes were completely located within the minimal amplicon (FIG. 6). EMSY gene, which has recently been reported as a candidate oncogene in breast and ovarian carcinomas was located at 76 Mb (13), close to but outside the minimal region of the amplification (FIG. 1a). Two methods were used to validate the digital karyotyping results. First, dual-color fluorescence in situ hybridization (FISH) was performed to validate the 11q13.5 amplification in these 3 tumors using a bacterial artificial chromosomes (BAC) probe located at the 11q13.5 minimal amplicon (FIG. 7) and a control probe located at 11q11 (21 Mb centromeric to the minimal amplicon). As shown in FIG. 1b, we found that all three amplified tumors defined by digital karyotyping showed distinct 11q13.5 amplification. Second, quantitative real-time PCR was performed to measure the DNA copy numbers at 12 loci flanking and within the amplicon including the EMSY gene in these three tumors (FIG. 1c). We found that increases in the DNA copy number were present at sub-chromosomal regions similar to the amplifications delineated by digital karyotyping. Furthermore, the fold of DNA copy number increase detected by quantitative PCR is at similar levels to that of digital karyotyping. A cutoff ratio of 2.2 was used to search for amplifications with greater than 97.5% confidence, and the delineated common region of amplification (from Loc220032 locus to FLJ23441 locus) was consistent with that derived from digital karyotyping.

To determine the frequency of the 11q13.5 minimal amplicon, we performed dual-color FISH on 211 paraffin-embedded ovarian tissue specimens using FISH probe located at the minimal amplicon and control FISH probe same as described above. The advantage of selecting the control FISH probe on the same chromosomal arm as the minimal amplicon is that it could facilitate distinguishing chromosome duplication from gene amplification, the latter involving smaller sub-chromosomal region (14). Using this method, we found 11q13.5 amplification in 16/121 (13.2%) high-grade serous carcinomas (FIG. 1b). In contrast, 11q13.5 gene amplification was not detected in any of 40 low-grade serous carcinomas, 14 serous borderline tumors, 19 benign cystadenomas and 17 normal ovaries. Thus, 11q13.5 amplification was detected exclusively in high-grade serous carcinomas. Among the 16 tumors with 11q13.5 amplification, 5 cases showed a homogeneous staining region (HSR) pattern, 3 cases showed a high level gain (>4.5 fold) and the remaining 8 tumors exhibited a moderate gain (between 2.5 and 4 fold). It should be noted that in addition to the 16 tumors with discrete amplification, we observed 11q polysomy in another 14 tumors based on an equal number of signals for both 11q13.5 and control probes. These tumors were not considered to have amplification specific to the 11q13.5 region in this study.

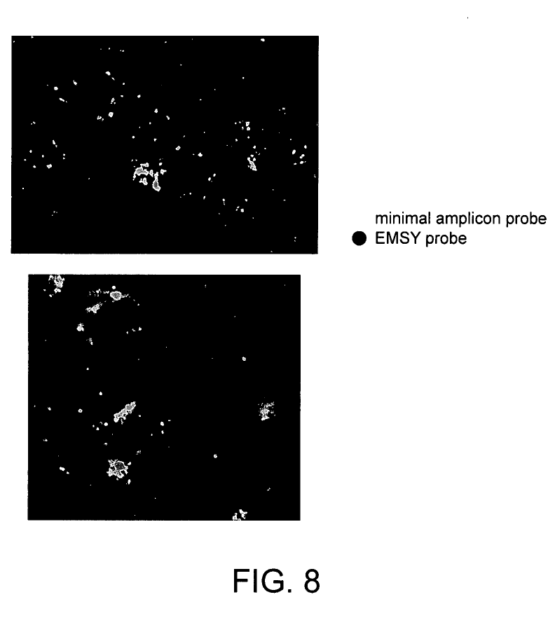

To further elucidate the physical relationship between EMSY and the minimal amplicon, we performed dual-color FISH in the same set of tumor samples using EMSY probe and same control probe. We found that EMSY was amplified in 12/117 (10.3%) high-grade serous carcinomas that were available for analysis. All 12 EMSY amplified carcinomas also demonstrated amplification at 11q13.5 minimal amplicon. Conversely, 4 of the 16 carcinomas that contained 11q13.5 minimal region amplification did not harbor EMSY amplification (FIG. 8). This result indicated that 11q13.5 minimal amplicon is more frequently amplified than its neighborhood region that contained EMSY gene in high-grade serous carcinomas.

Transcript Analysis of the 11q13.5 Minimal Amplicon

To identify the potential amplified oncogene within the 11q13.5 amplicon, we applied an approach based on the rationale that a tumor-driving gene, when amplified, almost always over-expresses to activate the tumorigenic pathway while co-amplified “passenger” genes that are unrelated to tumor development may or may not do so (14). Therefore, we searched for genes with both DNA amplification and transcript up-regulation in the same tumor samples. Ten high-grade ovarian carcinomas that contained 11q13.5 amplification and had their frozen tissues' available were analyzed by quantitative real-time PCR to assess mRNA levels in all the genes within the minimal amplicon. The same assay was also performed in 6 benign cystadenomas, 10 serous borderline tumors and 36 high-grade carcinomas that did not contain 11q13.5 amplification. Freshly brushed ovarian surface epithelium, which has been considered as an appropriate normal control, was used for normalization of gene expression (15). We used the Wilcoxon test to compute and compare the difference in gene expression levels between 11q13.5 amplified versus non-amplified high-grade carcinomas. We found that among the genes within the minimal amplicon, Rsf-1 (HBXAP) had the most significant difference (p=8.5×10⁻⁶) in expression levels between 11q13.5 amplified and non-amplified specimens. Furthermore, Rsf-1 was the only gene demonstrating consistent over-expression among the amplified tumors.

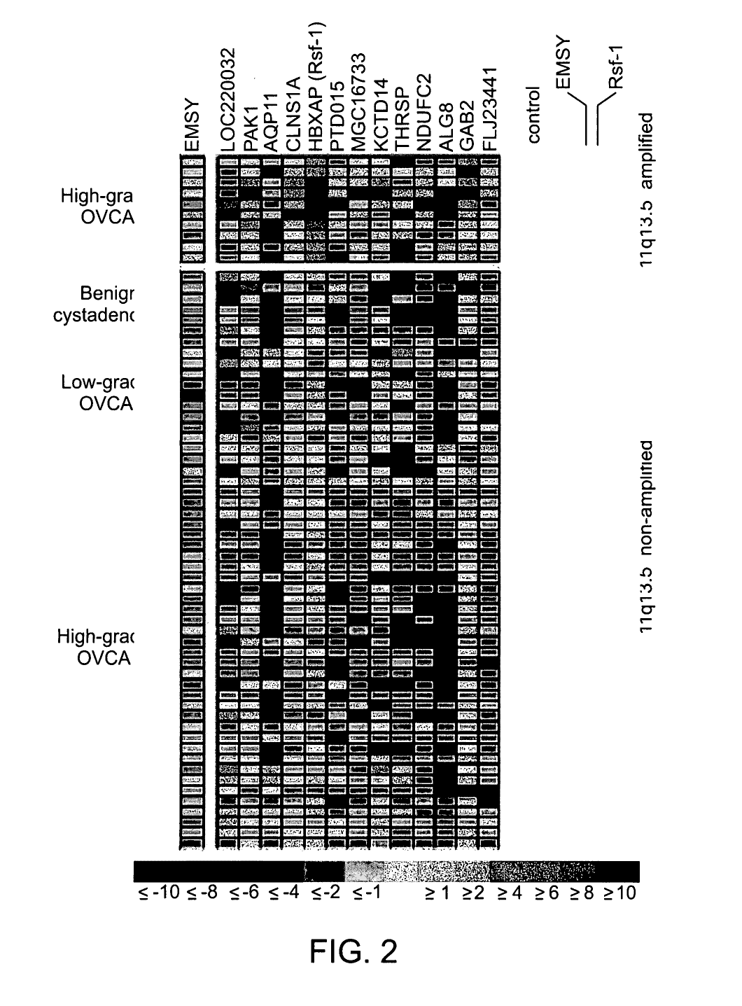

EMSY mRNA levels were also measured in parallel. We observed that although the EMSY gene was co-amplified in 8 of the tested samples, its RNA level was not consistently up-regulated as 5 of the tumors that harbored EMSY amplification down-regulated EMSY mRNA expression (FIG. 2).

Correlation of Rsf-1 Protein Over-Expression and Gene Amplification

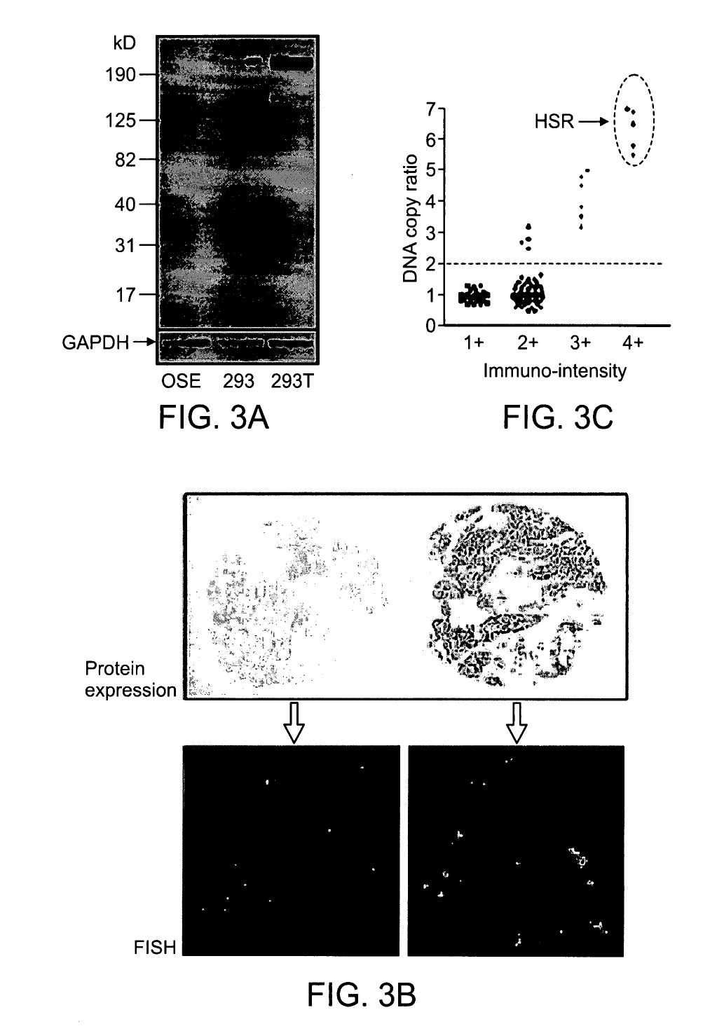

To demonstrate a more comprehensive correlation between Rsf-1 gene amplification and protein expression, we performed immunohistochemistry with an anti-Rsf-1 monoclonal antibody on the same panel of tissues used in FISH analysis. The specificity of the Rsf-1 monoclonal antibody has been demonstrated previously (16) and was independently confirmed in this study (FIG. 3a). Overall, there was a statistically significant correlation between Rsf-1 gene amplification and Rsf-1 immunoreactivity (p<0.001, Spearman correlation). We found that 11q13.5 non-amplified tumors demonstrated either weak (1+, 21% of tumors) or moderate (2+, 74% of tumors) Rsf-1 immunoreactivity (FIGS. 3b and c). In contrast, all the tumors with Rsf-1 amplification demonstrated an immunointensity of 2+-4+, with the most intense immunoreactivity (4+) found in those with a HSR pattern (n=5) and a strong immunoreactivity (3+, n=6) found in those with high-fold DNA gain (3-5 fold) (FIGS. 3b and c). Four tumors with mild gain (2-3.5 fold) in the Rsf-1 DNA copy number demonstrated moderate immunointensity (2+) and thus they were similar to the majority of the high-grade tumors without Rsf-1 amplification. This finding is likely attributed to the semi-quantitative nature inherent to immunohistochemistry in scoring mild to moderate immunointensity as such limitation in scoring Her2/neu immunointensity has been reported (17).

Clinical Significance of 11q13.5 Amplification and Rsf-1 Over-Expression

Amplification of the Rsf-1 locus and Rsf-1 over-expression were correlated with clinical outcome in patients with high-grade ovarian serous carcinoma. Since the FISH probe used to. assess 11q13.5 amplification contained the whole Rsf-1 coding region (FIG. 7), it allowed us using the same FISH data to analyze the clinical significance of Rsf-1 amplification. A total of 107 from 121 patients were available for survival analysis. The other 14 tumors harboring chromosome 11q polysomy were excluded in the analysis because polysomy was considered as duplication of chromosomal arms or large genomic segments and could not simply be grouped to either Rsf-1 amplified or non-amplified cases.

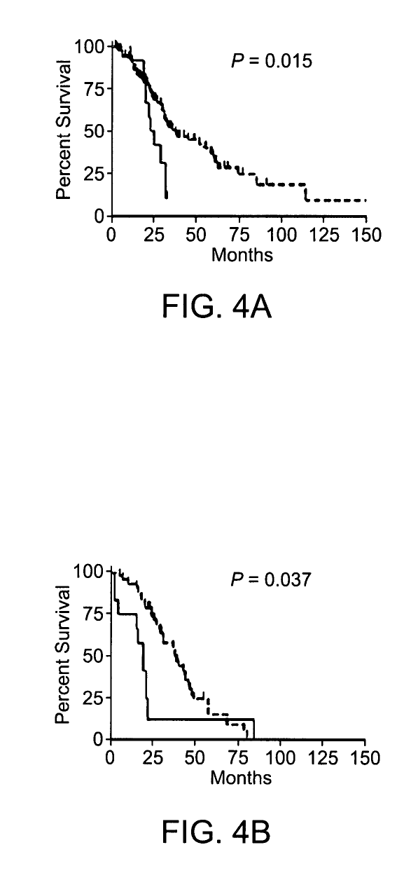

We found that all 107 patients had advanced stage high-grade serous carcinomas (the majority at FIGO stage III). Among them, 16 patients who had Rsf-1 amplification in their tumors had a shorter overall survival compared to those without amplification (p=0.015; Log rank test) (FIG. 4a). The median overall survival was 29 months (95% CI: 18.8-39.1 months) for the amplified group and 36 months (95% CI: 24.3-47.7 months) for the non-amplified group. Quantitative real-time PCR was also used to measure Rsf-1 mRNA in tumor cell pellets from 53 effusion samples that were not feasible for FISH analysis. An arbitrary cutoff of the expression level (>2.4 fold compared to normal ovarian surface epithelium) was used to assign specimens to either high expression (n=11) or low expression (n=42) groups. The results indicated that high levels of Rsf-1 mRNA expression (>2.4 fold) were correlated with poor outcome (p=0.037; Log rank test) (FIG. 4b) with median overall survival of 19 months (95% CI: 14.5-23.6) in patients with Rsf-1 mRNA over-expression and 38 months (95% CI: 28.3-47.8) in patients without Rsf-1 mRNA over-expression. Rsf-1 amplification and overexpression appeared as independent prognostic factors based on a multivariate analysis adjusted for patient age, clinical stage and differentiation status of tumor histology.

To further test if the clinical significance of Rsf-1 amplification and over-expression depended on the arbitrary cutoffs, we performed a survival analysis using continuous variables in a Cox proportional hazard model. The p-values assessed by a likelihood ratio test were 0.025 for FISH assay and 0.0013 for real-time PCR. These results further indicated that Rsf-1 amplification and over-expression were significantly correlated with poor survival, independent of the cutoffs.

In this study, we further compared the statistical significance in correlating gene amplification and overexpression and overall survival for both Rsf-1 and EMSY genes. For gene amplification, Rsf-1 had a more significant p value than that of EMSY (0.015 vs. 0.08). Similarly, for gene expression, Rsf-1 expression also had a more significant p value than EMSY expression (0.037 vs. 0.153).

Functional Analyses of Rsf-1 Expression

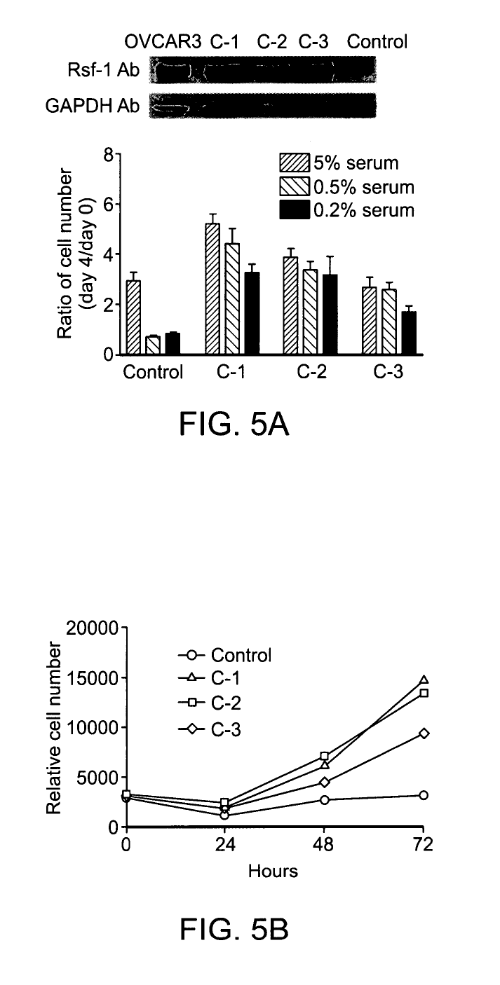

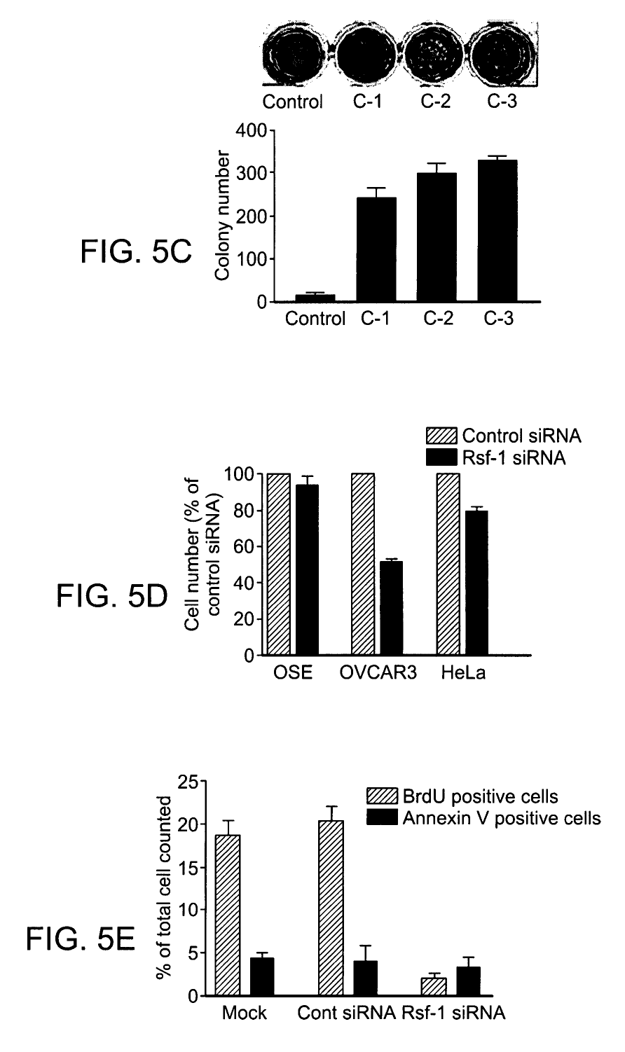

We therefore stably expressed the Rsf-1 gene in the non-neoplastic epithelial cells, RK3E, to assess if Rsf-1 expression induced transformation. RK3E cells have been used to evaluate the oncogenic potential of GL1, c-Myc, and mutant β-catenin and were considered as an appropriate in vitro model for oncogenic transformation( 8-20). Using quantitative real-time PCR, we found that ovarian serous carcinomas predominantly expressed the full-length form of the Rsf-1 gene (or HBXAPα), therefore RK3E cells were transfected with a vector expressing the full-length Rsf-1 and three independent clones were randomly selected for functional analyses. Western blot analysis confirmed the Rsf-1 expression in these clones (FIG. 5a). All the Rsf-1-expressing clones proliferated better at very low (0.2% and 0.5%) serum concentrations and showed a higher proliferative activity as compared to control RK3E cells (transfected with vector alone) based on increased cell numbers (FIGS. 5a and b) and BrdU incorporation. Rsf-1 -expressing clones grew anchorage independently as more colonies were observed in Rsf-1 expressing cells than in control cells (FIG. 5c). All the above differences were of statistical significance (p<0.001, t-test).

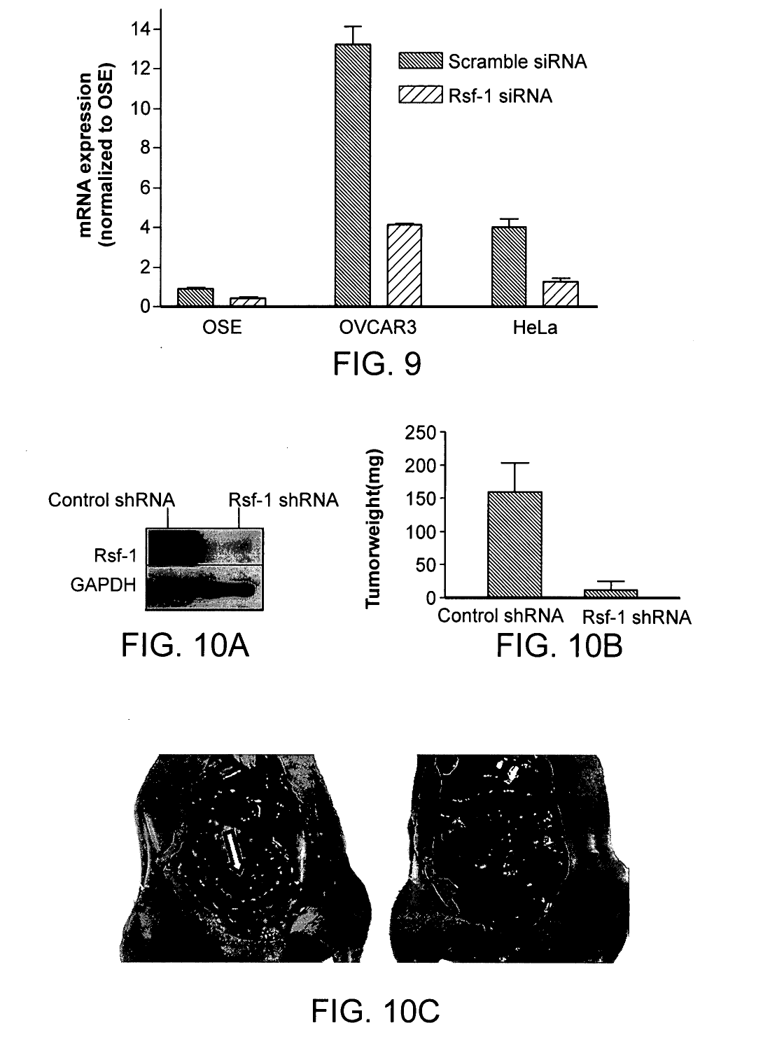

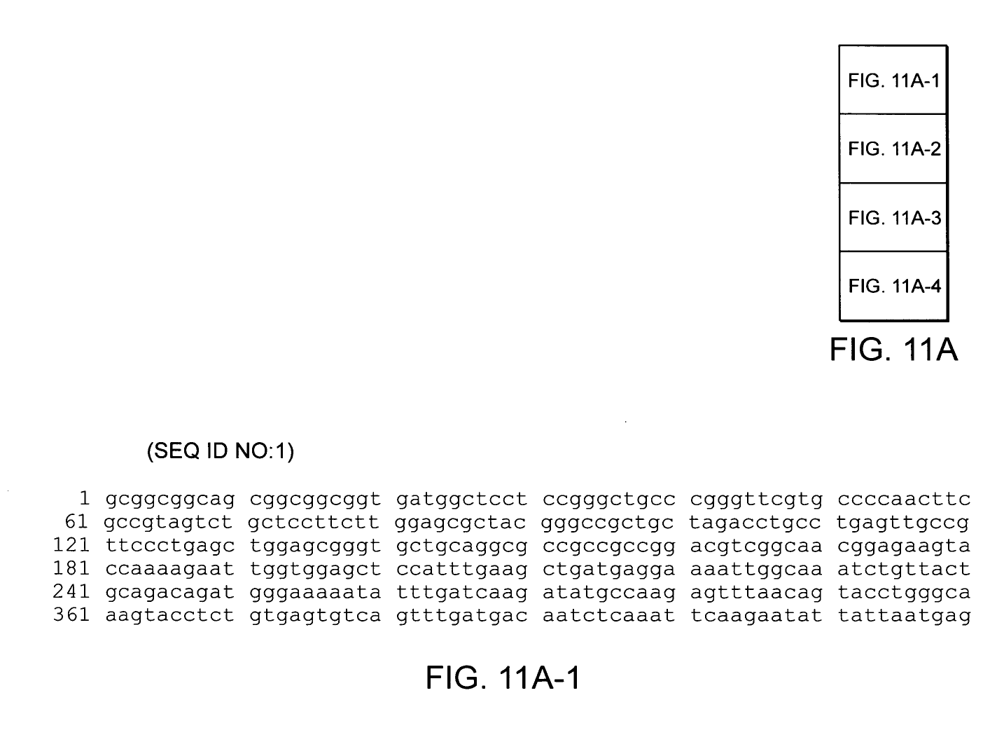

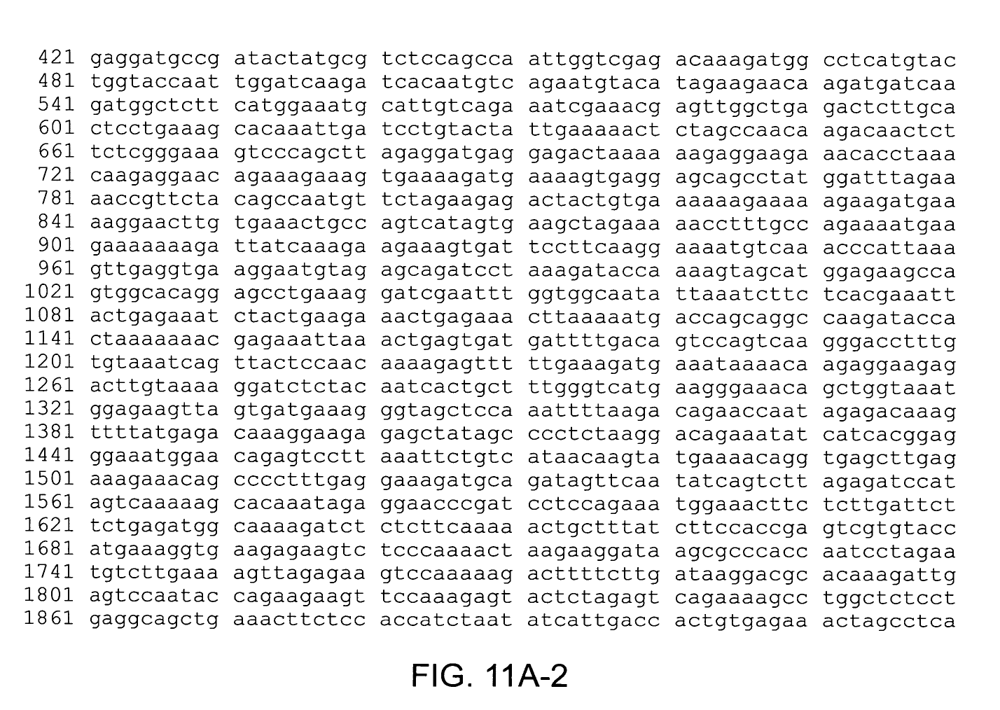

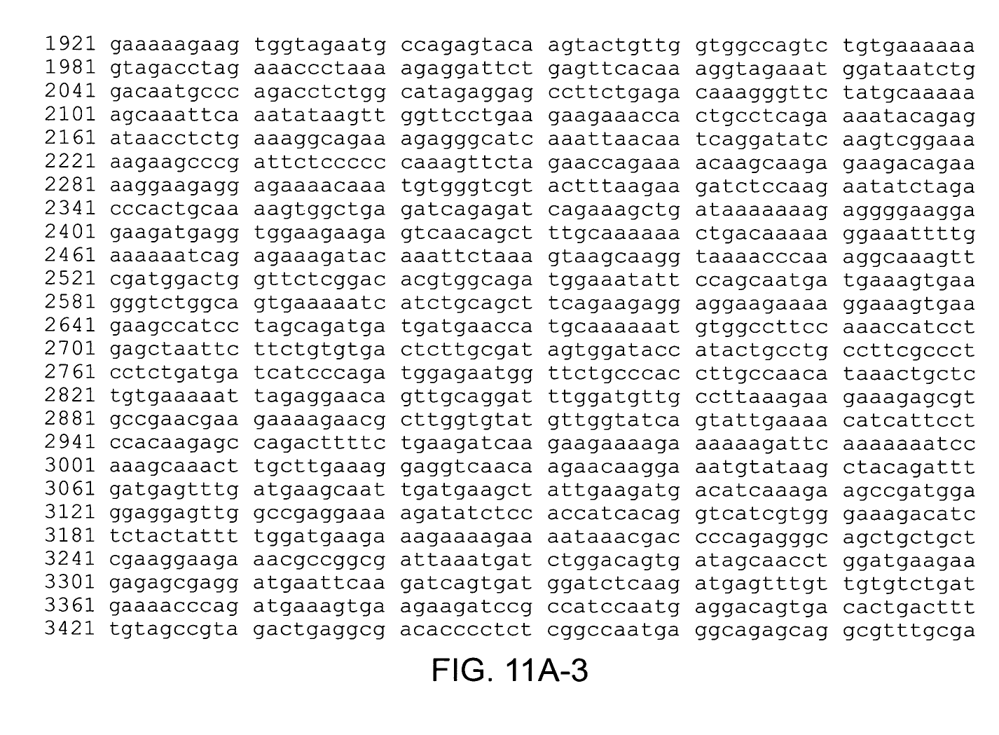

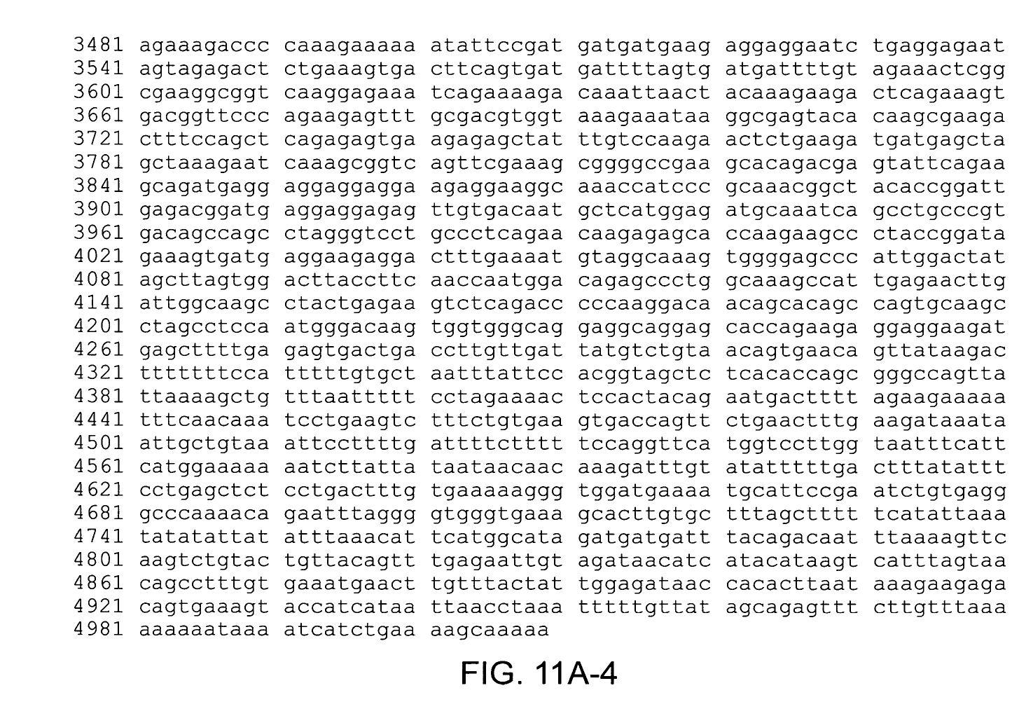

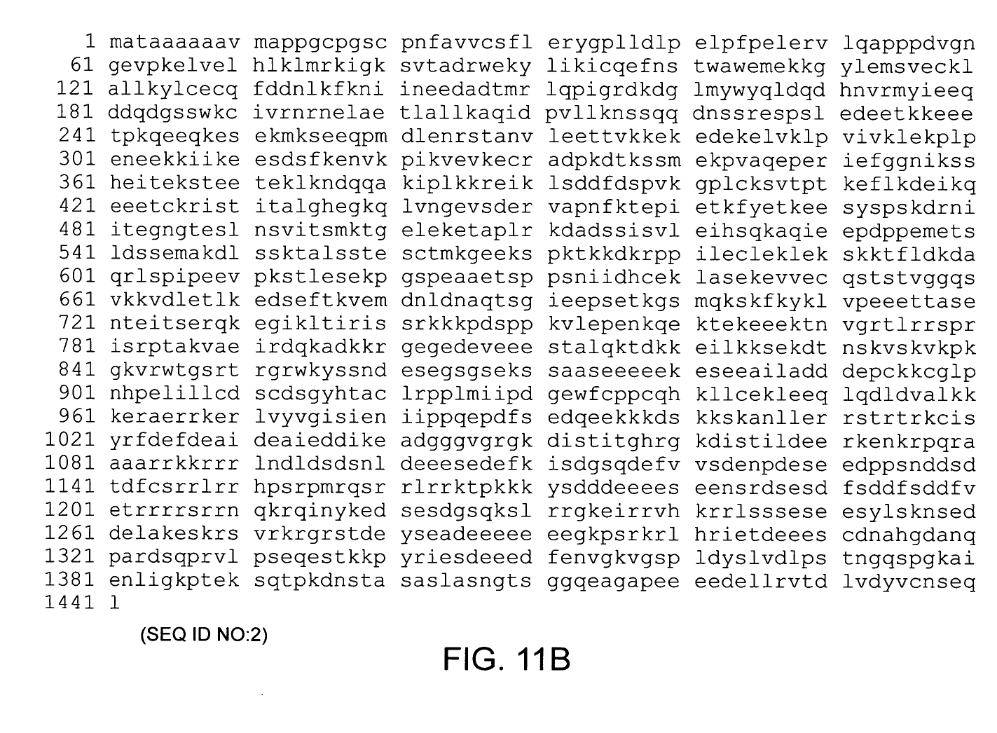

To further determine if Rsf-1 expression was essential for cell survival in cell lines that over-express Rsf-1, we used RNA interference to knockdown Rsf-1 expression in three cell lines including OVCAR3 cells (with Rsf-1 amplification and over-expression), Hela cells (without amplification but with Rsf-1 expression,) and ovarian surface epithelial cells (OSE; without Rsf-1 amplification or expression). The effect of Rsf-1 siRNA in suppressing Rsf-1 expression was confirmed by quantitative real time PCR (FIG. 9). Reduction of Rsf-1 protein expression significantly inhibited cell growth in Rsf-1-expressing cells including OVCAR3 and HeLa cells (FIG. 6b, p<0.001, t-test), with a more prominent inhibitory effect in Rsf-1 amplified OVCAR3 cells. In contrast, the same treatment did not affect cell growth in OSE cells, which had minimal Rsf-1 expression (p=0.26, t-test). The inhibition of cell growth after repressing Rsf-1 expression in OVCAR3 was likely a result of growth suppression as the percentage of BrdU-labeled cells was significantly decreased in Rsf-1 siRNA-treated as compared to control siRNA-treated OVCAR3 cells (FIG. 6c, p<0.001). In contrast, the percentage of apoptotic cells as measured by annexin V staining was similar between the Rsf-1 siRNA and control groups. To extend the findings of Rsf-1 gene knockdown in vitro, we transfected OVCAR3 cells with Rsf-1 shRNA before injecting the cells into nude mice. Western blot analysis demonstrated that Rsf-1 expression was substantially reduced in Rsf-1 shRNA transfected OVCAR3 cells as compared to the control shRNA transfected cells (FIG. 10). All mice injected with Rsf-1 shRNA treated OVCAR3 cells develop much smaller intra-abdominal xenograft tumors than the mice carrying control (scramble) shRNA transfected OVCAR3 cells (p<0.001, n=5).

Discussion

This study provides cogent evidences that amplification of Rsf-1 within the 11q13.5 minimal amplicon is involved in ovarian tumorigenesis based on a comprehensive study including molecular genetics, transcriptome analysis, clinical correlation and functional characterization. The frequency of 11q13.5 amplification in ovarian carcinoma detected in this study (13.2%) is similar to but slightly lower than that previously reported (17%) (13). This is likely due to more stringent criteria used for FISH analysis in the current Example. It should be noted that EMSY was located near the minimal amplicon delineated in the current study. EMSY functions as a BRAC2-interacting gene and was previously thought as a candidate oncogene for ovarian cancers (13). However, the oncogenic property of EMSY in ovarian tumor was not demonstrated in that study. Furthermore, our findings using a larger scale of ovarian tumor samples did not demonstrate a significant correlation of EMSY gene amplification and mRNA overexpression, a finding arguing against EMSY as the “driver” gene within the amplicon.

Based on our combined genetic and expression analyses, we have found that Rsf-1 is consistently over-expressed in all the amplified tumors examined. In addition to Rsf-1, several other genes close to Rsf-1 including CLNS 1 A, ALG8 and GAB2 were co-up-regulated in a subset of tumors with 11q13.5 amplification.

Recent in vitro studies have indicated that Rsf-1 plays a role in chromatin remodeling (16) and transcriptional regulation (21, 22) that may contribute to tumorigenesis. Rsf-1 has been shown to function as a histone chaperone while its binding partner, hSNF2H, possesses nucleosome-dependent ATPase activity. The Rsf-1/hSNF2H complex (or RSF complex) participates in chromatin remodeling by mobilizing nucleosomes in response to a variety of growth modifying signals and environmental cues. Such nucleosome remodeling is essential for transcriptional activation or repression (23, 24), DNA replication (25) and cell cycle progression (26). Recently, a growing body of evidences has accumulated to support a novel role of chromatin remodeling in cancer (27, 28). For example, mutations and deletions of a hSNF2H homolog, Brg1, were found in different tumor types (29) and furthermore, heterozygous deletion of Brg1 in mice resulted in a cancer-prone phenotype (30, 31).

### Example 2

### Correlation Between RSF-1 Expression and Clinical Outcome

Materials and Methods

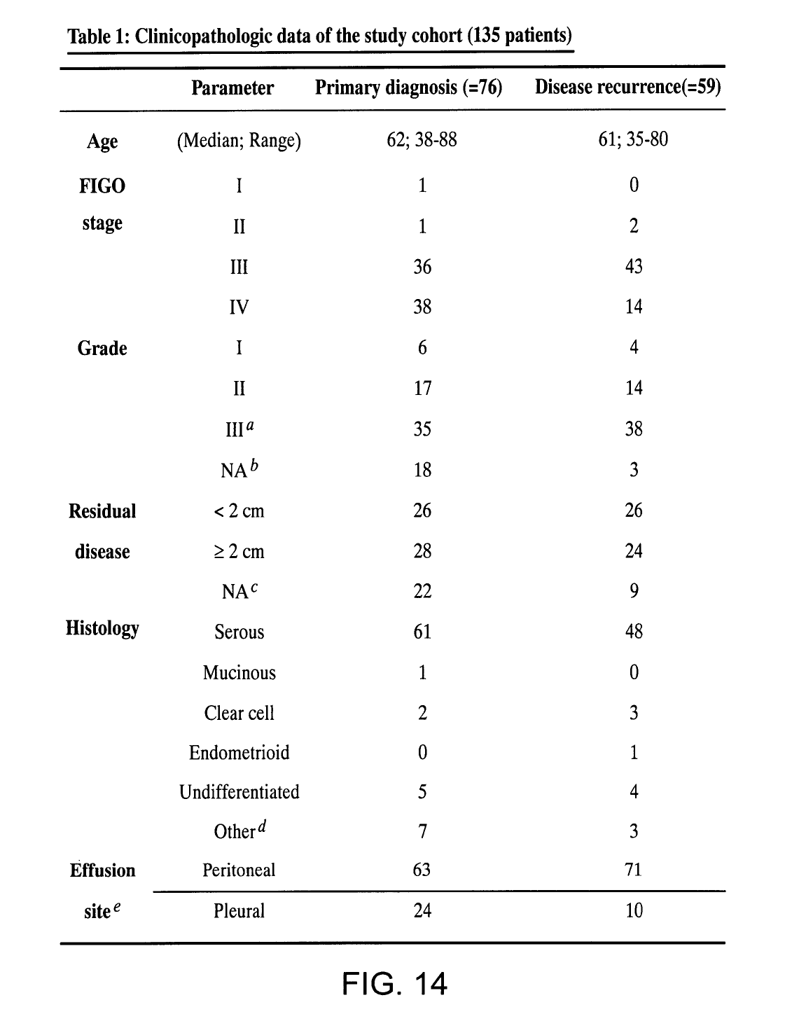

Study cohort: Specimens and relevant clinical data were obtained from the Department of Gynecologic Oncology, Norwegian Radium Hospital (Table 2 in FIG. 14). Informed consent was obtained according to national Norwegian and institutional guidelines. One hundred and sixty-eight fresh non-fixed malignant peritoneal (=134) and pleural (=34) effusions were obtained from 121 patients diagnosed with epithelial (predominantly serous) ovarian carcinoma (151 effusions), 4 patients with serous carcinoma of the fallopian tube (5 effusions), and 10 patients with primary peritoneal serous carcinoma (12 effusions) (total=135 patients). Due to their closely linked histogenesis and phenotype, these tumors will all be referred to as ovarian carcinomas henceforth. Effusions were submitted for routine diagnostic purposes to the Department of Pathology, Norwegian Radium Hospital during 1998-2002. Cell blocks were prepared using the thrombin clot method (35).

Diagnoses were established by evaluation of smears and sections from cell blocks, and further confirmed using immunocytochemistry with broad antibody panels against carcinoma, mesothelial and leukocyte epitopes, as previously detailed (35-36). Surgical specimens were reviewed in order to confirm the diagnosis, histologic type and grade.

Immunocytochemistry: Rsf-1 protein expression was analyzed using a mouse monoclonal anti-Rsf-1 antibody clone 5H2/E4, (Upstate, Lake Placid, N.Y.) with an optimal dilution previously determined (1: 2,000). The specificity of the Rsf-1 monoclonal antibody was previously demonstrated (37) and was independently confirmed in our recent study (33). Pretreatment consisted of microwave oven antigen retrieval in EDTA buffer. Visualization was achieved using the EnVision TM+ peroxidase syste (DakoCytomation, Glostrup, Denmark). Positive controls consisted of an ovarian carcinoma shown to be positive in a pilot study. Negative controls were stained with an antibody for isotypic mouse myeloma protein.

Staining was scored by an experienced cytopathologist (BD) who was blinded to the patient clinical data. Nuclear expression was interpreted as positive. Staining extent was scored on a scale of 0-4, corresponding to percentage of immunoreactive tumor cells of 0%, 1-5%, 6-25%, 26-75% and 76-100%, respectively. Staining intensity was scored as negative (=0), weak (=1) or strong (=2). A staining score ranging from 0-8 was calculated by multiplying the staining extent and intensity values, with a resulting low (0-4) or high (36-39) expression score for each specimen. At least 500 tumor cells were scored, when present (>90% of cases). No specimen contained less than 100 tumor cells.

Statistical analysis: Statistical analysis was performed applying the SPSS-PC package (Chicago, Ill.). Probability of <0.05 was considered statistically significant. Complete clinicopathologic data were available for the majority of patients (Table 1). Survival data were available for all 135 patients. Analysis of the association between Rsf-1 protein and clinicopathologic parameters was analyzed using the two-sided Chi-square test.

Progression-free survival (PFS) and overall survival (OS) were calculated from the date of diagnosis to the date of recurrence/death or last follow-up. Univariate survival analyses of PFS and OS were executed using the Kaplan-Meier method and log-rank test.

For this analysis, expression categories were grouped in order to include a sufficient number of patients in each category (negative/weak vs. strong expression for intensity, 0-3 vs. 4 for staining extent, low vs. high score as detailed above). For patients with more than one effusion, expression in the first specimen was analyzed. Multivariate analyses for OS and PFS were performed using the Cox proportional hazard model.

Results

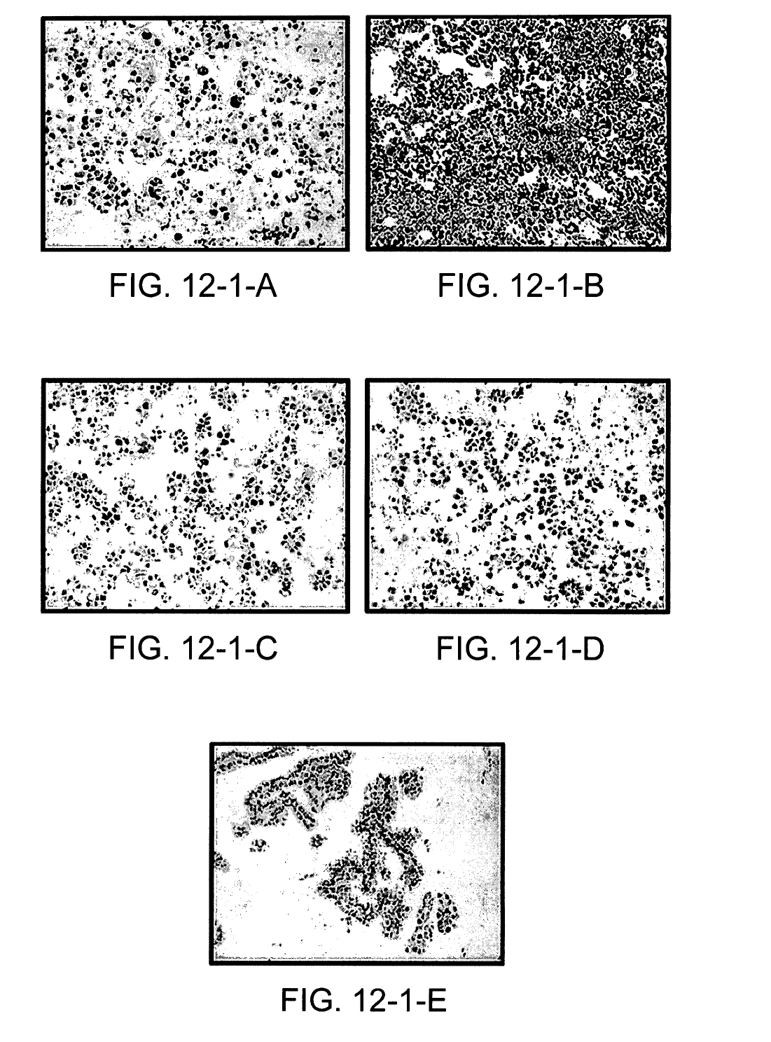

Rsf-1 immunoreactivity was found in carcinoma cells in 157/168 (93%) effusions (70 weakly and 87 strongly positive specimens, FIG. 12). The percentage of Rsf-1 positive cells in the 157 specimens was as follows: 1-5%: 14 specimens; 6-25%: 15 specimens; 26-75%: 51 specimens; 76-100%: 77 specimens. Cancer cells in a given specimen tended to have generally homogenous staining intensity. Reactive cells (leukocytes and mesothelial cells) were negative in the majority (>95%) of cases (FIG. 12). Serial specimens from the same patient showed comparable staining intensity and percentage of stained cells.

In analysis of the association between Rsf-1 expression and clinicopathologic parameters, we found that specimens from patients diagnosed with FIGO stage IV disease had higher staining score compared to stage III tumors (p=0.008). In analysis of staining extent and intensity separately, a trend for association was seen between FIGO stage IV disease and more intense immunoreactivity (p=0.052). No association was seen with tumor grade, histologic type (serous vs. non-serous types), the extent of residual disease, patient age (≦60 vs. >60 years) or effusion site (peritoneal vs. pleural).

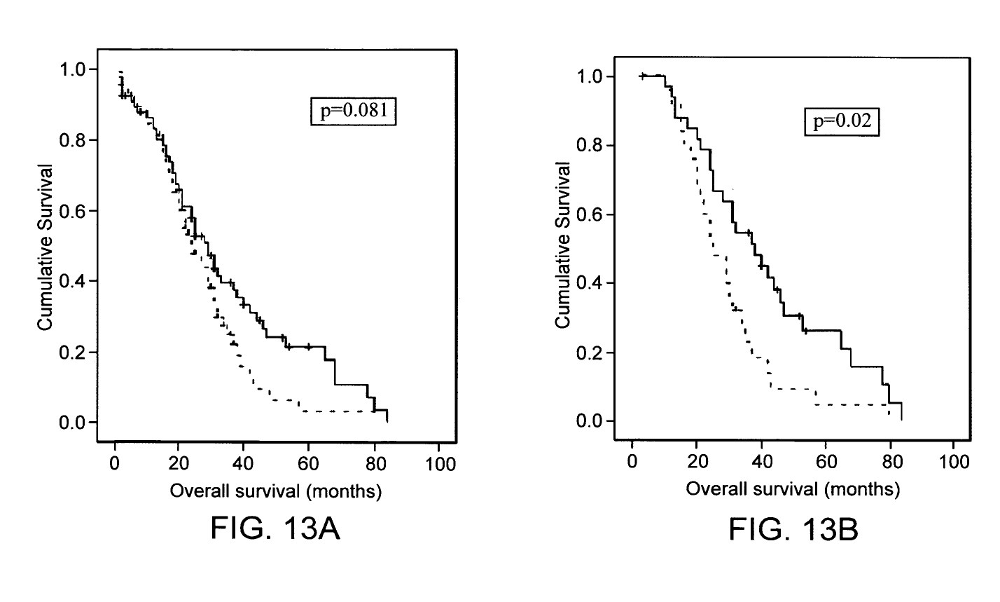

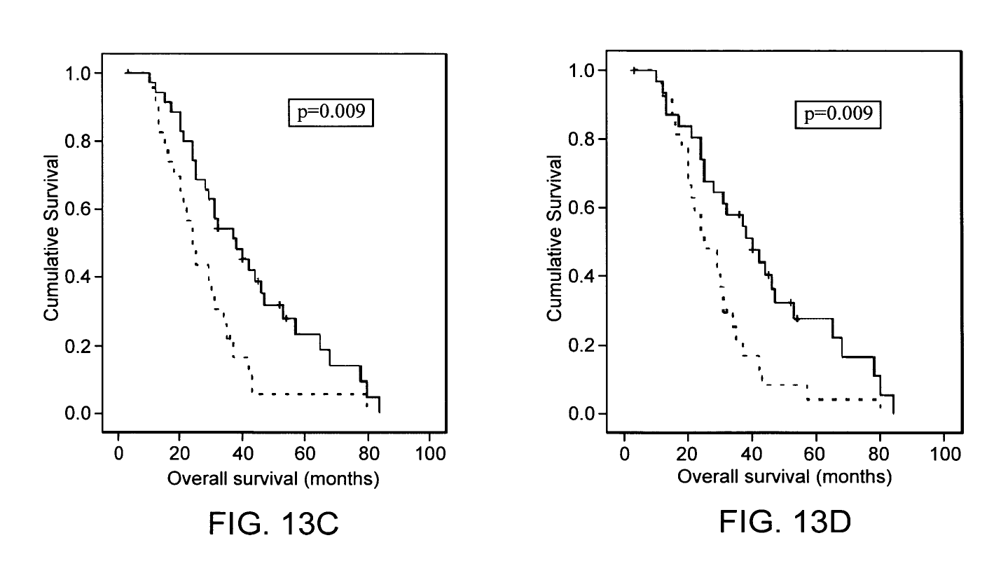

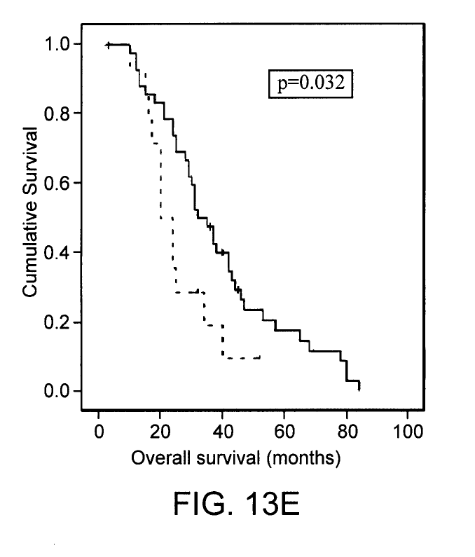

Follow-up ranged from 1 to 85 months (mean=26 months, median=23 months). At the last follow-up, 102 patients were dead of disease, 24 were alive with disease and 9 were with no evidence of disease. In univariate survival analysis of the entire cohort, a trend for poor OS for patients with effusions showing high Rsf-1 score was found (p=0.081, FIG. 13). When patients with pre- and post-chemotherapy specimens were analyzed separately, higher Rsf-1 score significantly correlated with poor OS for 59 patients with post-chemotherapy (disease recurrence) specimens (p=0.02, FIG. 13B). A similar correlation with shorter OS was seen in separate analysis of staining extent (staining in >75% of carcinoma cells) and intensity (p=0.009 for both, FIGS. 13C, 13D). FIGO stage (IV vs. III) was the only clinical parameter that correlated with OS in this group (p=0.032, FIG. 13E). Diagnosis at FIGO stage IV also correlated with significantly worse PFS (p<0.001, median=4 vs. 9 months, mean=3 vs. 11 months, data not shown).

Stronger expression of Rsf-1 showed only a trend for worse PFS (p=0.057). In Cox analysis for OS, Rsf-1 expression (p=0.022 for staining extent and intensity, p=0.045 for score) and FIGO stage (p=0.035) were independent predictors of poor survival.

Discussion

As in other cancer types, gene amplification is one of the major mechanisms mediating oncogene activation and thereby dysregulated growth in ovarian cancer. Chromosome regions that have been shown to be amplified in cell lines or clinical specimens of ovarian carcinoma include 17q21-23 (38), 19q13.1-q13.2 (39), 3q26, 8q24, and 20q13 (40) or 20q12-13 (41). Specific genes that have been reported as amplified in ovarian cancer are Ki-ras (42), HER2/neu (43-44), INT-2 (44), AKT2 (45), cyclin E (46-47), Cdk2 (47), SEI-I (48), L-Myc (49), EMSY (50), STK15/BTAK (51), TGIF2 (52) and TPD52 (53).

The previous experiment demonstrated that Rsf-1 is amplified in ovarian cancer, and that a significant correlation exists between Rsf-1 amplification and overexpression, based on FISH and semi-quantitative real-time PCR, respectively, and worse overall survival in patients with advanced stage ovarian carcinoma. In the this example, we analyzed the clinical role of Rsf-1 protein in ovarian cancer patients with malignant effusions.

Genetic material in human cells is stored in the form of chromatin, which consists of nucleoprotein complexes containing DNA and protein. DNA is wrapped around an octamer core of histones whose position and density are regulated through active chromatin remodeling by ATP-dependent complexes (54). The degree of association between histones and DNA, and thereby DNA transcription, is regulated by ubiquitination, methylation, acetylation, phosphorylation and poly-ADP ribozylation (54).

Chromatin-remodeling complexes in mammalian cells include the SNF2-like subfamily, the ISWI-like subfamily, and the CHD subfamily (54). SNF2-like subfamily members regulate transcription though interactions with the retinoblastoma protein and BRCA1.

Mutations or repression of these genes have been shown in a variety of cancers, including carcinomas, as well as in different human syndromes (54-55).

RSF is a chromatin-remodeling complex that is composed of two subunits, hSNF2H and p325 (Rsf-1). The recently cloned (37) RSF was initially found in HeLa cells and is involved in formation of RNA polymerase II complexes (25. A truncated form of RSFp325 codes for the hepatitis B virus transcription repressor HBXAP (37, 57-58). Amplification of the Rsf-1 gene in occurs selectively in highgrade ovarian carcinoma, but is absent in less aggressive ovarian tumors and benign ovaries (33). In this example, we found ubiquitous expression of Rsf-1 protein in ovarian carcinoma cells in effusions, with rare expression in benign reactive cells, suggesting that Rsf-1 expression is a cancer-specific event also in effusions. The majority of the above-listed genes that are amplified in ovarian carcinoma have not been studied for their prognostic role. Expression of others has not been found to correlate with disease outcome, as evidenced by data regarding INT-2 amplification (59), K-ras mutation (60) and p-AKT expression (61) in primary ovarian carcinoma. Data for cyclin E are controversial (62-66).

In the present study, we found that Rsf-1 expression score is associated with more advanced (FIGO stage IV) disease at diagnosis and shows a trend for poor overall survival. Separate analysis of patients with post-chemotherapy specimens showed that Rsf-1 staining extent, staining intensity and expression score are all independent markers of poor survival in patients. The data regarding Rsf-1 provide further evidence supporting the clinical rationale in analyzing the expression of molecular markers in metastatic carcinoma cells for their association with aggressive clinical behavior and for the need to separate specimens obtained at primary diagnosis from those associated with disease recurrence in survival analyses.

This Example provides new evidence that Rsf-1 immunoreactivity in effusion samples correlates with statistically significant shorter survival in ovarian cancer patients with disease recurrence specimens.

### Example 3

### Multilocation Survival Analysis Based on RSF-1 Expression

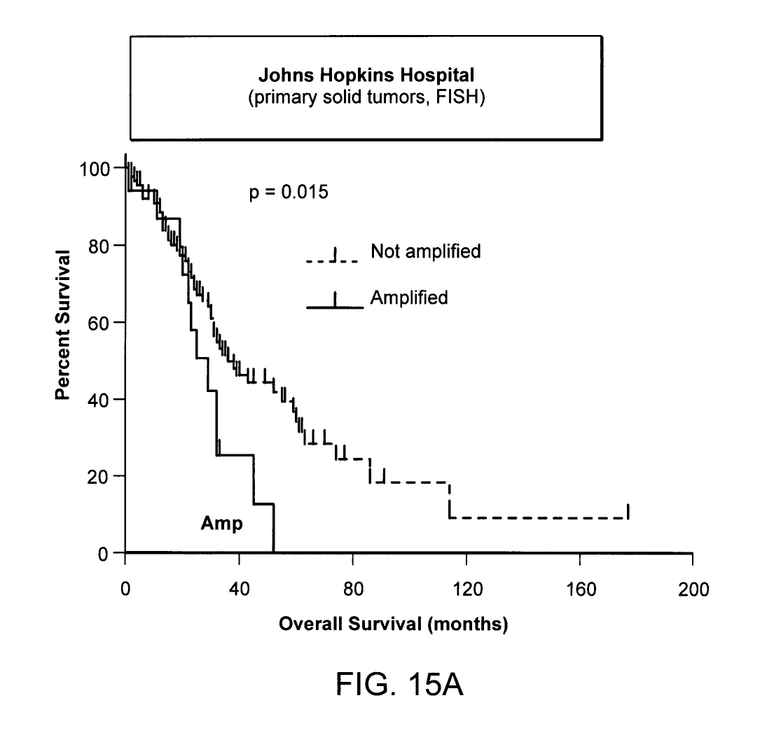

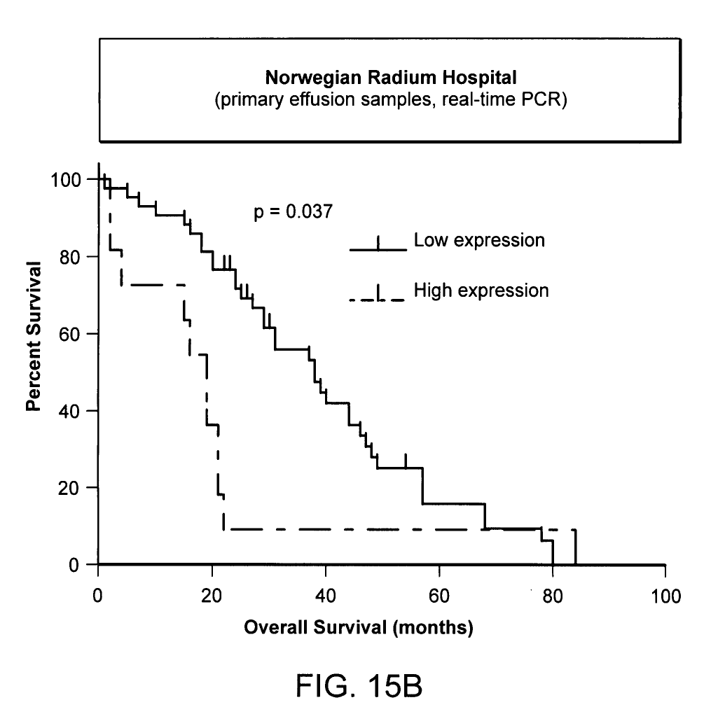

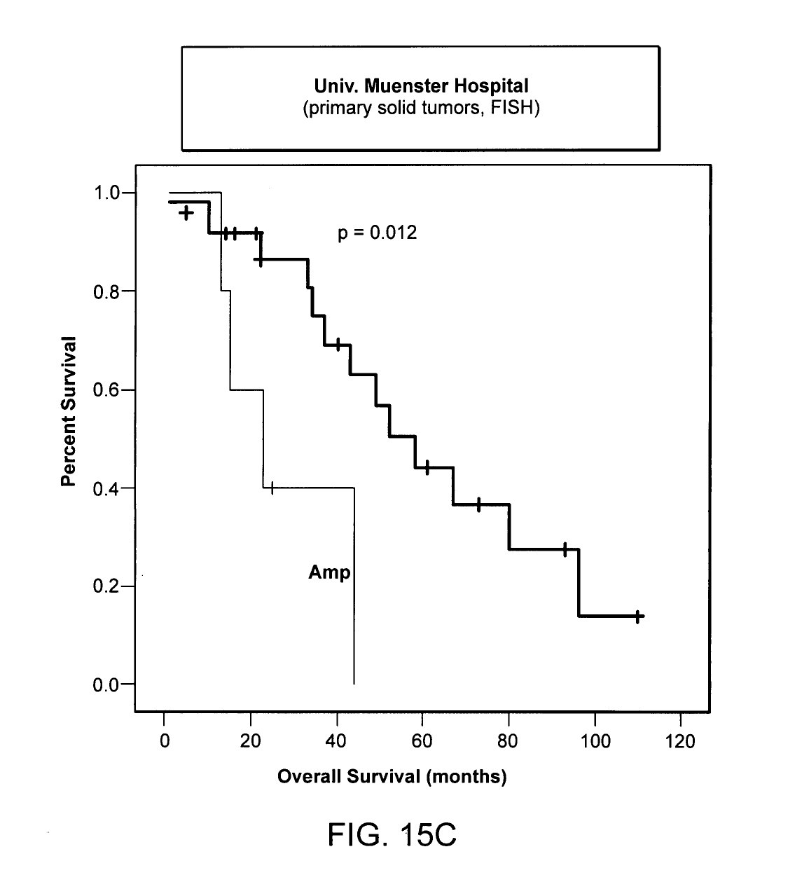

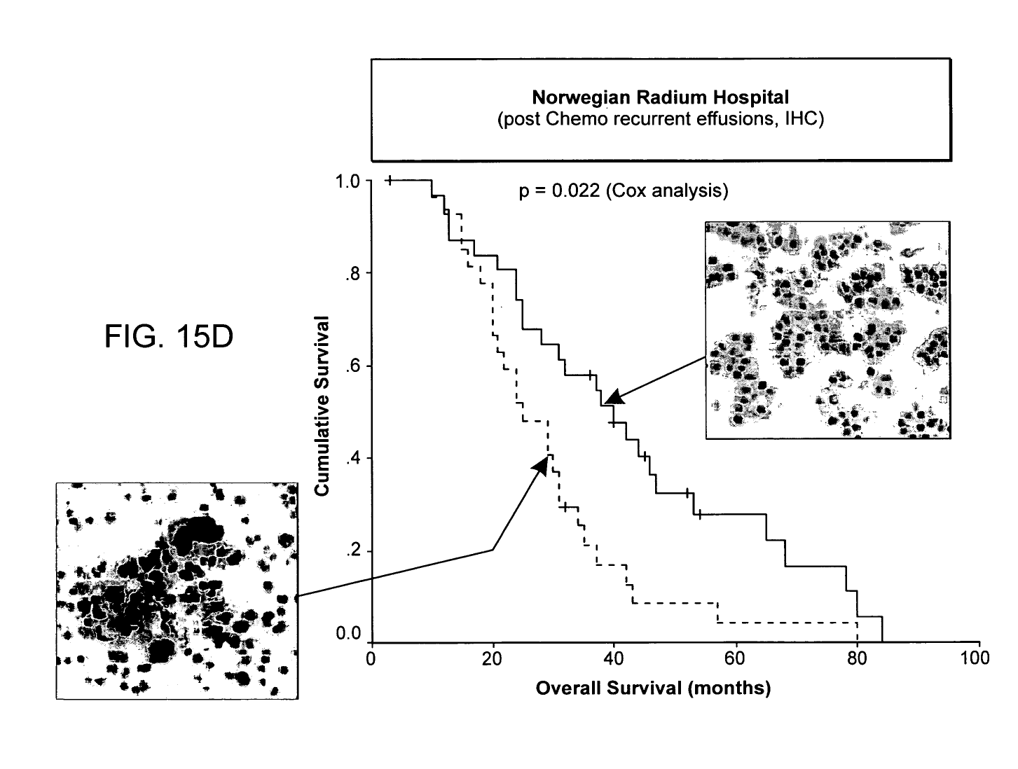

The survival of subjects diagnosed with cancer was monitored as a function of time. The length of survival was plotted against the level of RSF-1 protein or nucleic acid expression. FIG. 15A demonstrates significantly shortened survival in subjects with RSF-1 overexpression in primary solid tumors as determined using FISH. FIG. 15B demonstrates significantly shortened survival of subjects with high levels of RSF-1 expression in primary effusion samples as detected by real time PCR. FIG. 15C depicts significantly shortened survival in subjects with RSF-1 overexpression in primary solid tumors as determined using FISH. FIG. 15D demonstrates significantly shortened survival of subjects with RSF-1 overexpression in post Chemo recurrent effusions as detected by IHC.

The results of this multilocation study demonstrate that increased RSF-1 nucleic acid or polypeptide expression is indicative of shortened survival and therefore, RSF-1 is a valuable prognostic marker.

### REFERENCES

1. Meltzer, P. S., Kallioniemi, A. & Trent, J. M. (2002) in The genetic basis of human cancer, eds. Vogelstein, B. & Kinzler, K. W. (McGraw-Hill, New York), pp. 93-113.
2. Courjal, F., Louason, G., Speiser, P., Katsaros, D., Zeillinger, R. & Theillet, C. (1996) Int J Cancer 69, 247-53.
3. Slamon, D. J., Godolphin, W., Jones, L. A., Holt, J. A., Wong, S. G., Keith, D. E., Levin, W. J., Stuart, S. G., Udove, J., Ullrich, A. & et al. (1989) Science 244, 707-12.
4. Cheng, J. Q., Godwin, A. K., Bellacosa, A., Taguchi, T., Franke, T. F., Hamilton, T. C., Tsichlis, P. N. & Testa, J. R. (1992) Proc Natl Acad Sci U S A 89, 9267-71.
5. Wu, R., Lin, L., Beer, D. G., Ellenson, L. H., Lamb, B. J., Rouillard, J. M., Kuick, R., Hanash, S., Schwartz, D. R., Fearon, E. R. & Cho, K. R. (2003) Am J Pathol 162, 1603-10.
6. Wang, T. L., Maierhofer, C., Speicher, M. R., Lengauer, C., Vogelstein, B., Kinzler, K. W. & Velculescu, V. E. (2002) Proc Natl Acad Sci U S A 99, 16156-61.
7. Wang, T. L., Diaz, L. A., Jr., Romans, K., Bardelli, A., Saha, S., Galizia, G., Choti, M., Donehower, R., Parmigiani, G., Shih le, M., Iacobuzio-Donahue, C., Kinzler, K. W., Vogelstein, B., Lengauer, C. & Velculescu, V. E. (2004) Proc Natl Acad Sci U S A 101, 3089-94.
8. Di, C., Liao, S., Adamson, D. C., Parrett, T. J., Broderick, D. K., Shi, Q., Lengauer, C., Cummins, J. M., Velculescu, V. E., Fults, D. W., McLendon, R. E., Bigner, D. D. & Yan, H. (2005) Cancer Res 65, 919-24.
9. Hu, M., Yao, J., Cai, L., Bachman, K. E., van den Brule, F., Velculescu, V. & Polyak, K. (2005) Nat Genet 37, 899-905.
10. Boon, K., Eberhart, C. G. & Riggins, G. J. (2005) Cancer Res 65, 703-7.
11. Buckhaults, P., Zhang, Z., Chen, Y. C., Wang, T. L., St Croix, B., Saha, S., Bardelli, A., Morin, P. J., Polyak, K., Hruban, R. H., Velculescu, V. E. & Shih le, M. (2003) Cancer Res 63, 4144-9.
12. Shih, I. M., Yu, J., He, T. C., Vogelstein, B. & Kinzler, K. W. (2000) Cancer Res 60, 1671-6.
13. Hughes-Davies, L., Huntsman, D., Ruas, M., Fuks, F., Bye, J., Chin, S. F., Milner, J., Brown, L. A., Hsu, F., Gilks, B., Nielsen, T., Schulzer, M., Chia, S., Ragaz, J., Cahn, A., Linger, L., Ozdag, H., Cattaneo, E., Jordanova, E. S., Schuuring, E., Yu, D. S., Venkitaraman, A., Ponder, B., Doherty, A., Aparicio, S., Bentley, D., Theillet, C., Ponting, C. P., Caldas, C. & Kouzarides, T. (2003) Cell 115, 523-35.
14. Hogarty, M. D. & Brodeur, G. M. (2002) in The genetic basis of human cancer, eds. Vogelstein, B. & Kinzler, K. w. (McGraw-Hill, New York), pp. 115-128.
15. Zorn, K. K., Jazaeri, A. A., Awtrey, C. S., Gardner, G. J., Mok, S. C., Boyd, J. & Birrer, M. J. (2003) Clin Cancer Res 9, 4811-8.
16. Loyola, A., Huang, J.-Y., LeRoy, G., Hu, S., Wang, Y.-H., Donnelly, R. J., Lane, W. S., Lee, S.-C. & Reinberg, D. (2003) Mol. Cell. Biol. 23, 6759-6768.
17. Hicks, D. G. & Tubbs, R. R. (2005) Hum Pathol 36,250-61.
18. Ruppert, J. M., Vogelstein, B. & Kinzler, K. W. (1991) Mol Cell Biol 11, 1724-8.
19. Foster, K. W., Ren, S., Louro, I. D., Lobo-Ruppert, S. M., McKie-Bell, P., Grizzle, W., Hayes, M. R., Broker, T. R., Chow, L. T. & Ruppert, J. M. (1999) Cell Growth Differ 10, 423-34.
20. Kolligs, F. T., Hu, G., Dang, C. V. & Fearon, E. R. (1999) Mol Cell Biol 19, 5696-706.
21. Shamay, M., Barak, O., Doitsh, G., Ben-Dor, I. & Shaul, Y. (2002) J Biol Chem 277, 9982-8.
22. Shamay, M., Barak, O. & Shaul, Y. (2002) Genomics 79, 523-9.
23. Holstege, F. C., Jennings, E. G., Wyrick, J. J., Lee, T. I., Hengartner, C. J., Green, M. R., Golub, T. R., Lander, E. S. & Young, R. A. (1998) Cell 95, 717-28.
24. Vignali, M., Hassan, A. H., Neely, K. E., Workman, J. L. (2000) Mol Cell Biol. 20, 1899-910.
25. Flanagan, J. F., Peterson, C.L. (1999) Nucleic Acids Res. 27, 2022-8.
26. Cosma, M. P., Tanaka, T. & Nasmyth, K. (1999) Cell 97, 299-311.
27. Wolffe, A. P. (2001) Oncogene 20, 2988-90.
28. Klochendler-Yeivin, A., Muchardt, C. & Yaniv, M. (2002) Curr Opin Genet Dev 12, 73-9.
29. Wong, A. K., Shanahan, F., Chen, Y., Lian, L., Ha, P., Hendricks, K., Ghaffari, S., Iliev, D., Penn, B., Woodland, A. M., Smith, R., Salada, G., Carillo, A., Laity, K., Gupte, J., Swedlund, B., Tavtigian, S. V., Teng, D. H. & Lees, E. (2000) Cancer Res 60, 6171-7.
30. Klochendler-Yeivin, A., Fiette, L., Barra, J., Muchardt, C., Babinet, C. & Yaniv, M. (2000) EMBO Rep 1, 500-6.
31. Guidi, C. J., Sands, A. T., Zambrowicz, B. P., Turner, T. K., Demers, D. A., Webster, W., Smith, T. W., Imbalzano, A. N. & Jones, S. N. (2001) Mol Cell Biol 21, 3598-603.
32. United States Cancer Statistics 2002
33. Shih leM, Sheu J J, Santillan A, et al. Proc Natl Acad Sci U S A. 2005;102:14004-14009.
34. Bibbo M (editor). Comprehensive Cytopathology. Philadelphia: W.B. Saunders, 1991 (p. 543).
35. Davidson B, Risberg B, Kristensen G, et al. Virchows Arch. 1999;435:43-49.
36. Davidson B, Nielsen S, Christensen J, et al. Am J Surg Pathol. 2001;25:1405-1412.
37. Loyola A, Huang J-Y, LeRoy G, et al. Mol Cell Biol. 2003;23:6759-6768.
38. Watanabe T, Imoto I, Kosugi Y, et al. Gynecol Oncol. 2001;81:172-177.
39. Thompson F H, Nelson M A, Trent J M, et al. Cancer Genet Cytogenet. 1996;87:55-62.
40. Sonoda G, Palazzo J, du Manoir S, et al. Genes Chromosomes Cancer. 1997;20:320-328.
41. Tanner M M, Grenman S, Koul A, et al. Clin Cancer Res. 2000;6:1833-1839.
42. Fukumoto M, Estensen R D, Sha L, et al. Cancer Res. 1989;49:1693-1697.
43. Zhang X, Silva E, Gershenson D, Hung M C. Oncogene. 1989;4:985-989.
44. Hruza C, Dobianer K, Beck A, et al. Eur J Cancer. 1993;29A:1593-1597.
45. Bellacosa A, de Feo D, Godwin A K, et al. Int J Cancer. 1995;64:280-285.
46. Courjal F, Louason G, Speiser P, Katsaros D, Zeillinger R, Theillet C. Int J Cancer. 1996;69:247-253.
47. Marone M, Scambia G, Giannitelli C, et al. Int J Cancer. 1998;75:34-39.
48. Tang T C, Sham J S, Xie D, et al. Cancer Res. 2002;62:7157-7161.
49. Wu R, Lin L, Beer D G, et al. Am J Pathol. 2003;162:1603-1610.
50. Hughes-Davies L, Huntsman D, Ruas M, et al. Cell. 2003;115:523-535.
51. Zhou H, Kuang J, Zhong L, et al. Nat Genet. 1998;20:189-193.
52. Imoto I, Pimkhaokham A, Watanabe T, Saito-Ohara F, Soeda E, Inazawa J. Biochem Biophys Res Commun. 2000;276:264-270.
53. Byrne J A; Balleine R L, Fejzo M S, et al. 2005;117:1049-1054.
54. Cho K S, Elizondo L I, Boerkoel C F. Curr Opin Genet Dev. 2004; 14:308-315.
55. Wong A K, Shanahan F, Chen Y, et al. Cancer Res. 2000;60, 6171-6177.
56. LeRoy G, Orphanides G, Lane W S, Reinberg D. Science. 1998;282: 1900-1904.
57. Shamay M, Barak O, Doitsh G, Ben-Dor I, Shaul Y. J Biol Chem. 2002;277: 9982-9988.
58. Shamay M, Barak O, Shaul Y. Genomics. 2002;79;523-529.
59. Medl M, Sevelda P, Czerwenka K, et al. Gynecol Oncol. 1995;59:321-326.
60. Cuatrecasas M, Erill N, Musulen E, Costa I, Matias-Guiu X, Prat J. Cancer. 1998;82:1088-1095.
61. Wang Y, Kristensen G B, Helland A, Nesland J M, Borrsen-Dale A L, Holm R. Am J Clin Pathol. 2005;124:392-401.
62. Blegen H, Einhom N, Sjovall K, et al. Int J Gynecol Cancer. 2000;10:477-487.
63. Rosenberg E, Demopoulos R I, Zeleniuch-Jacquotte A, et al. Hum Pathol. 2001;32:808-813.
64. Sui L, Dong Y, Ohno M, Sugimoto K, Tai Y, Hando T, Tokuda M. Gynecol Oncol. 2001;83:56-63.
65. Farley J, Smith L M, Darcy K M, et al. Cancer Res. 2003;63:1235-1241.
66. Milde-Langosch K, Hagen M, Bamberger A M, Loning T. Int J Gynecol Pathol. 2003;22:168-174.
67. Davidson B, Risberg B, Reich R, Berner A. Clin Lab Med. 2003;23:729-754.
68. Davidson B. Diagn Cytopathol. 2004;31:246-254.
69. Davidson B, Alejandro E, Florenes V A, et al. Cancer. 2004;100:2139-2147.
70. Elloul S, Bukholt Elstrand M, Nesland J M, et al. Cancer. 2005;103:1631-1643.
71. Stevens E V, Raffeld M, Espina V, et al. Cancer. 2005;103:2313-2319.
   Incorporation by Reference

The contents of all references, patents, pending patent applications and published patents, cited throughout this application are hereby expressly incorporated by reference.

Equivalents

Those skilled in the art will recognize, or be able to ascertain using no more than routine experimentation, many equivalents to the specific embodiments of the invention described herein. Such equivalents are intended to be encompassed by the following claims.

What is claimed is:

1. A method for detecting cancer in a subject comprising:
detecting the amount of the nucleic acid of SEQ ID NO:1, or a fragment thereof, in a biological sample from the subject;
wherein overexpression of SEQ ID NO:1 indicates that the subject has cancer.

2. The method of claim 1, wherein the amount of the nucleic acid of SEQ ID NO:1 or a fragment thereof, is detected by FISH.

3. The method of claim 1, wherein the biological sample is from a solid tumor.

4. The method of claim 3, wherein the solid tumor is selected from a liver, breast, lung, or ovarian tumor.

5. The method of claim 1, wherein the biological sample is an ovarian tissue sample.

6. A method for detecting cancer in a subject comprising:
detecting the amount of the polypeptide of SEQ ID NO:2, or a fragment thereof, in a biological sample from the subject;
wherein overexpression of SEQ ID NO:2 indicates that the subject has cancer.

7. The method of claim 6, wherein the amount of the polypeptide of SEQ ID NO:2 or a fragment thereof, is detected by immunohistochemistry.

8. The method of claim 6, wherein the biological sample is from a solid tumor.

9. The method of claim 8, wherein the solid tumor is selected from a liver, breast, lung, or ovarian tumor.

10. The method of claim 6, wherein the biological sample is an ovarian tissue sample.

11. A method for determining the prognosis of a subject having cancer, comprising:
determining the amount of the nucleic acid of SEQ ID NO:1 present in a biological sample from the subject;
wherein overexpression of the nucleic acid of SEQ ID NO:1 as compared to a control is indicative of poor prognosis.

12. The method of claim 11, wherein the cancer is ovarian cancer.

13. A method for determining the prognosis of a subject having cancer, comprising:
determining the amount of the amino acid of SEQ ID NO:2 present in a biological sample from the subject;
wherein overexpression of SEQ ID NO:2 as compared to a control is indicative of poor prognosis.

14. The method of claim 13, wherein the cancer is ovarian cancer.

15. A method for identifying a compound for the treatment or prevention of cancer comprising:
contacting a cell with a test compound;
determining if the test compound inhibits the expression of the nucleic acid of SEQ ID NO:1;
thereby identifying a compound for the treatment or prevention of cancer.

16. A method for identifying a compound for the treatment or prevention of cancer comprising:
contacting a cell with a test compound;
determining if the test compound inhibits the expression or activity of the polypeptide of SEQ ID NO:2;
thereby identifying a compound for the treatment or prevention of cancer.

17. A method of treating or preventing cancer in a subject comprising:
administering to a subject a compound that inhibits the expression or activity of a nucleic acid of SEQ ID NO:1 or a polypeptide of SEQ ID NO:2;
thereby treating the subject.

18. The method of claim 17, wherein the cancer is selected from the group consisting of ovarian, lung, liver and breast cancer.

19. A kit comprising a nucleic acid molecule, for use in determining the amount of the nucleic acid of SEQ ID NO:1 in a sample and instructions for use.

20. A kit comprising an antibody, or fragment thereof, for use in determining the amount of the polypeptide of SEQ ID NO:2 in a sample and instructions for use.
